(19) United States
(12) Patent Application Publication
(10) Pub. No.: US 2013/0189282 A1
(43) Pub. Date: Jul. 25, 2013

(54) METHODS FOR ISOLATING AND USING A SUBSET OF CD8 T-CELLS THAT ARE RESISTANT TO CYCLOSPORIN

(71) Applicant: Raymond M. Johnson, Indidnapolis, IN (US)

(72) Inventor: Raymond M. Johnson, Indidnapolis, IN (US)

(73) Assignee: Indiana University Research and Technology Corpora, Indianapolis, IN (US)

(21) Appl. No.: 13/811,806

(22) PCT Filed: Jul. 25, 2011
(86) PCT No.: PCT/US11/45212

§ 371 Date: Apr. 8, 2013

Related U.S. Application Data
(60) Provisional application No. 61/367,127, filed on Jul. 23, 2010.

(51) Int. Cl. C12N 5/0783

(52) U.S. Cl. CPC C12N 5/0636
USPC 424/172.1; 435/325; 435/375; 435/7.1; 514/16.5; 514/44 A

(57) ABSTRACT

Utilizing a novel T cell culture system based on allogeneic epithelial antigen presenting cells (semi-professional APC), a cyclosporin-resistant CD8 T cell clone with minimal cytolytic capability was isolated. Derivation of the novel alloantigen-specific CD8 T cell clones involved previous priming with an allogeneic skin graft, implying expansion of this T cell subset during transplant rejection. Characterization and comparison of the cyclosporin and rapamycin-resistant CD 8 T cell clone with typical cyclosporin-sensitive CD 8 T cells suggests that it is a member of a CD8 T cell subset with a unique cell surface phenotype and novel TCR activation pathways, and that these unique CD8 T cell clones reflect the immunobiology of chronic rejection within the non-hematopoetic microenvironments of solid organs and vascular walls. These cells express the aryl-hydrocarbon receptor. T-cells of this type are referred to herein as CD8bm12-1 T-cells.

### PRIORITY CLAIM

This application claims the benefit of U.S. provisional patent application No. 61/367,127 filed on Jul. 23, 2010 and incorporated herein by reference in its entirety.

### STATEMENT REGARDING FEDERALLY SPONSORED RESEARCH OR DEVELOPMENT

This invention was made with government support under NIH-K08-A1052128-01 awarded by the National Institutes of Health. The United States Government has certain rights in the invention.

### FIELD OF THE INVENTION

Some aspects of this invention relate to identifying, and using cells of the mammalian immune system including as subset of CD8 T cells that are resistant to both cyclosporin and rapamycin.

### BACKGROUND

Current surgical techniques and immunosuppressive medications have improved the survival of solid organ transplant patients. One year survival rates are currently >80% and acute rejection is no longer the major cause of organ failure and death. Instead, late organ failure due to chronic rejection has become the major hurdle impeding long-term survival. 50% of lung and heart transplant patients have graft attrition at 5 & 10 years post transplant respectively (1). Roughly 50% of lung transplant patients have bronchiolitis obliterans syndrome (BOS) at year five (2), while ˜50% of surviving cardiac transplant patients have chronic allograft vasculopathy (CAV) by year 8 (3). Chronic allograft rejection similarly plagues the long term survival of bone marrow transplant patients (4). Understanding the pathophysiology of chronic rejection is critical toward developing new strategies for therapeutic interventions to improve long term survival in transplant patients.

The pathophysiology driving chronic rejection is not well understood. There are mouse models for BOS based on heterotopic tracheal transplantation (5) and bone marrow transplantation (6). Data from the heterotopic trachea transplant model have elegantly shown that allogeneic airway epithelial cells are the primary target of the T cell response (7), and shown that both CD4 and CD8 T cells can mediate rejection (5). A recent rat orthotopic lung allo-transplant model incorporating cyclosporin A (CsA) and rapamycin treatment reproduced the histopathology of BOS (8); similarly a recent study showed that rapamycin was ineffective in preventing CAV in a rat cardiac transplant model (9). The animal model data are consistent with the clinical experience that current immunosuppressive drugs, including mTOR inhibitors (10), may not effectively inhibit T cell subsets mediating chronic rejection.

Intensive investigations in murine CAV models have provided insight into the effector T cell subset mediating chronic allograft rejection. In one mouse CAV model primed CD8 T cells were sufficient to cause vasculopathy in completely MHC-mismatched aortic grafts. Intimal proliferation was independent of allo-MHC class I on the aortic graft, implying CD8 recognition of allo-MHC class II molecules (11). Similarly in a nude mouse model, adoptive transfer of naïve C57BL/6 CD8 T cells was sufficient to cause CAV in MHC class II-mismatched bm12 cardiac allografts; again implying CD8 recognition of allo-MHC class II molecules. In that study CAV was dependent on IFN-γ but not perform or fas ligand (12). Tbet-deficient mice have a vigorous costimulation-blockade resistant CAV that is mediated by IL-17-secreting CD8 T cells (13). In a critical study, cyclosporin A prevented vasculopathy caused by CD4 T cells, but was ineffective in preventing vasculopathy caused by CD8 T cells (14). Effector CD8 T cells rather than effector CD4 T cells appear to have a calcineurin-independent pathway for T cell activation during chronic allograft rejection.

In solid organ transplants chronic allograft rejection is characterized by progressive fibrosis rather than acute inflammation and necrosis seen in acute rejection. Given the distinct pathologies associated with chronic allograft rejection and CAV, there is a need to better understand the pathophysiology of chronic allograft rejection and develop new strategies for therapeutic interventions to prolong solid organ and bone marrow transplant patients' survival. Some aspects and embodiments of the instant invention address this need.

### BRIEF DESCRIPTION OF THE SEQUENCES

SEQ ID NO. 1 is a predicted I-A^{bm12}—specific peptide which, according to the subject disclosure, binds a CD8T cell epiptope on MHC class I K^{b}.

### BRIEF DESCRIPTION OF THE FIGURES

FIG. 1. Flow cytometry of CD4 vs. CD8 on polyclonal T cell populations from mouse #3 and mouse #4 after culturing immune splenocytes from C57BL/6 mice (I-A^{b}) previously primed with bm12 skin grafts on Bm12.4 epithelial cells (I-A^{bm12}; MHC class II mismatch) as the alloantigen presenting cell; result- yielded exclusively CD8 T cells rather than the conventional CD4 T cells that were expected in the MHC class II-mismatched bm12 mouse model.

FIG. 2A. Bar graphs of pg ml⁻¹ of IFN-gamma produced by CD8 alloreactive T-cell clones recognizing either Bm1.11 (white bars) or Bm12.4 (black bars); *=p value<0.001. Demonstrates the specificity of the T cell clones.

FIG. 2B. Bar graphs of pg ml⁻¹ of IFN-gamma produced by CD8 alloreactive T-cell clones in media (hatched bar), or activated by CL.7 control cells (white bars) or CL.7I-A^{bm12} cells (black bars); *=p value<0.001. Demonstrates that CD8bm12-1 & CD8bm12-2 recognize MHC class II I-A^{bm12} directly.

FIG. 3A. Flow cytometry data measuring cell surface phenotypes of CD8bm1.

FIG. 3B. Flow cytometry data measuring cell surface phenotypes of CD8bm12-1.

FIG. 3C. Flow cytometry data measuring cell surface phenotypes of CD8bm12-2.

FIG. 4A. Plot of % specific lysis, vs. Effector target ratio as measured for CD8bm1, CD8bm12-1, and CD8bm12-2 with allo-epithelial target cells (Bm1.11 for CD8bm1; Bm12.4 for CD8bm12-1 & CD8bm12-2). Demonstrates that the novel CD8 T cell clones CD8bm12-1 & CD8bm12-2 recognizing MHC class II I-A^{bm12} have a non- or minimally-cytolytic phenotype.

FIG. 4B. Plot of % specific lysis vs. Effector: Target ratio measured for two fibroblast-derived alloreactive CD8 T cell clones with H-2^{d} bearing CL.7 fibroblast targets. Demonstrates cytolytic capabilities of conventional CD8 T cells.

FIG. 5A. Plot of proliferation of CD8bm1 (▪) and CD8bm12-1 () activated by immobilized anti-CD3 over the full range of cyclosporin A tested.

FIG. 5B. Plot of IL-2 production of CD8bm1(▪) and CD8bm12-1 () activated by immobilized anti-CD3 over the full range of cyclosporin A tested.

FIG. 5C. Plot of IL-10 production of CD8bm1 (▪) and CD8bm12-1 () activated by immobilized anti-CD3 over the full range of cyclosporin A tested.

FIG. 5D. Plot of IFN-gamma production of CD8bm1 (▪) and CD8bm12-1 () activated by immobilized anti-CD3 over the full range of cyclosporin A tested.

FIG. 6A. Plot of the proliferation of CD8bm12-1 cells exposed to only media (unactivated cells), and CD8bm12-1 cells activated by exposure to immobilized anti-CD3 antibody in the presence of 30 μM, 10 μM, 3 μM, or 0 μM CH-223191 (aryl hydrocarbon receptor antagonist). **=pvalue<0.005; ***=pvalue<0.0005.

FIG. 6B. Plot of the proliferation of CD8bm12-1 cells exposed to only media (unactivated cells), and CD8bm12-1 cells activated by exposure to immobilized anti-CD3 antibody in the presence of 30 μM, 10 μM, 3 μM, or 0 μM CH-223191. **=pvalue<0.005; ***=pvalue<0.0005

FIG. 7. Plot of the proliferation of CD8bm12-1 cells exposed to only media (unactivated cells), and CD8bm12-1 cells activated by exposure to immobilized anti-CD3 antibody in the presence of either 90 μM or 30 μM rapamycin. ***=pvalue<0.0005.

### SUMMARY

Some aspects of the invention include methods for isolating a subset of CD8 T-cells, comprising the steps of: transplanting an allorgan into an animal and monitoring the animal; recovering CD8 T-cells from the animal, culturing said CD8 T-cells on a layer of semi-professional antigen presenting cells; and selecting the lymphocyte cells that proliferate on the layer of semi-professional antigen presenting cells. In some embodiments the semi-professional antigen presenting cells are selected from the group consisting of smooth muscle cells, endothelial cells and epithelial cells. Some embodiments further include the steps of: contacting said CD8 T-cells with cyclosporine, wherein the concentration of cyclosporin in contact with said cells is sufficient to inhibit the growth of most types of CD8 T-cells; and harvesting the CD8 T-cells that proliferate in the presence of cyclosporin.

Some methods for isolating a subset of CD8 T-cells further include the steps of contacting said CD8 T cells with an antibody wherein the antibody selectively binds to IL-18r1. And still other embodiments include the steps of testing said CD8 T-cells to determine if the CD8 T-cells express at least one gene selected from the group consisting of: Mest, Padi2, Ahr, Klhl6, Rasgrp3, Klhi30, Trib2, Rab17, Prkcz, PLCγ2, scin, Pla2g4a, CD7, I118r1, IL-17a, IL-17f, Sgk3, GprlS, Pls3 and Zfp187. In some embodiments the CD8 T-cell is CD8bm12-1 or an equivalent cell type. The proliferation and/or viability of these cells may be effected by contacting them with compounds that interact with the aryl hydrocarbon receptor or components of the novel CD8bm12-1 T cell receptor signalling cascade.

Other aspects of the invention include methods of collecting cyclosporin and/or rapamycin resistant CD8 T-cells, comprising the steps of: providing a sample from an animal wherein the sample includes CD8 T-cells, wherein the animal has undergone an allorgan transplant, supplying an antibody, wherein said antibody selectively binds to at least one protein selected from the group consisting of anti-CD7 antibody or anti-Il-18r1 antibody; contacting the sample with said antibody.

Still other aspects of the invention include methods for collecting cyclosporin and/or rapamycin resistant CD8 T-cells, comprising the steps of: providing a sample from an animal wherein the sample includes CD8 T-cells, wherein the animal has undergone an allorgan transplant; supplying an antibody, wherein said antibody selectively binds to Il18r1; and contacting the sample with said antibody. In some embodiments the method further includes the step of: recovering the CD8-T cells that were contacted with said antibody to Il18r1 and wherein the CD8 T-cells that are recovered are bound to an antibody to Il18r1. In some embodiments the CD8 T-cell is CD8bm12-1 or an equivalent cell type.

Yet other aspects of the invention include methods for selecting for compounds that regulate a subset of CD8 T-cells which are resistant to cyclosporin and/or rapamycin, comprising the steps of: providing a population of CD8 T-cells wherein said CD8 T-cells express Il18r1 and aryl hydrocarbon receptor (Ahr); contacting said CD8 T-cells with at least one compound; and measuring the effect of the compounds on the population of said CD8 T-cells. The CD8 T-cells may be contacted with the compound either in vitro or in vivo. Compounds that help to regulate this subset of CD8 T-cells may act, at least in part, by inhibiting the expression or function of at least one of the genes selected from the group consisting of: Mest, Padi2, Ahr, Klh16, Rasgrp3, Klhi30, Trib2, Rab17, Prkcz, PLCγ2, scin, Pla2g4a, CD7, Il18r1, IL-17a, IL-17f, Sgk3, Gpr15, Pls3 and Zfp187. In some embodiments the compounds are selected from a group of compounds that preferentially binds to at least one gene product encoded by at least one of the genes selected from the group consisting of: Mest, Padi2, Ahr, Klh16, Rasgrp3, Klhi30, Trib2, Rab17, Prkcz, PLCγ2, scin, Pla2g4a, CD7, Il18r1, IL-17a, IL-17f, Sgk3, Gpr15, P1s3 and Zfp187.

Other aspects of the invention include methods for treating allograft rejection, comprising the steps of: identifying a patient wherein said patient has undergone an allorgan transplant; providing at least one therapeutically effective dose of at least one compound that inhibits the proliferation of a subset of CD8 T-cells, wherein said subset of CD8 T-cells expresses Il18r1 and/or the Ahr and is resistant to levels of cyclosporin that inhibit most other subsets of CD8 T-cells; and treating the patient with the at least one therapeutically effective dose of the at least one compound. In some embodiments the patient is either a human or an animal. In some embodiments the compound that is used to effect the activity of the subset of CD8 T-cells is a small molecule that inhibits the expression of at least one gene selected from the group consisting of: Mest, Padi2, Ahr, Klh16, Rasgrp3, Klhi30, Trib2, Rab17, Prkcz, PLCγ2, scin, Pla2g4a, CD7, Il18r1, IL-17a, IL-17f, Sgk3, Gpr15, Pls3 and Zfp187. While in still other embodiments the compound is an interference RNA. And in other embodiments the compound is at least one selected compound selected from the group consisting of; polyclonal antibodies, or monoclonal antibodies, wherein the antibodies preferentially bind to the cyclosporin resistant CD8 T-cells. In still another embodiment the compound is humanized anti CD7 antibody. While in still another embodiment the compound is humanized anti IL-18 receptor antibody.

Still other aspects of the invention include methods of inhibiting or at least reducing the progression of chronic allograft rejection comprising the steps of contacting cyclosporin resistant CD8 T-cells with at least one compounds that inhibits the proliferation of this subset of cells, compounds suitable for inhibiting these cells include compounds that inhibit at least one gene product selected from the group consisting of antibodies such as anti-CD7 antibody, small molecules and the like.

In some embodiments the compound used to regulate chronic allograft rejection is an antibody that preferentially binds to CD7 such antibodies may include, but are not limited to, the human mouse chimeric CD7 monoclonal antibody SDZCHH380. Still other embodiments include methods of selecting for at least one molecule that inhibits or at least slows the progression of chronic allograft rejection, comprising the step of providing at a population of cyclosporin resistant CD8 T-cells; contacting the cells with at least one compound and measuring the compound's effect on the proliferation and or survival of said cyclosporin resistant CD8 T-cells.

Some embodiments include methods for treating allograft vasculopathy or chronic allograft rejection, comprising the steps of: identifying a patient that has undergone an allorgan or an allograft transplant; providing at least one therapeutically effective dose of at least one compound that inhibits the proliferation of a subset of CD8 T-cells, wherein said subset of CD8 T-cells expresses Il18r1 and is resistant to levels of cyclosporin and/or rapamycin that inhibits the proliferation of most other subsets of CD8 T-cells; and treating the patient with the at least one therapeutically effective dose of said at least one compound. In some embodiments the patient treated for allograph vaculopathy is either an animal or a human.

In some embodiments that compounds used to treat the patient is as leas one compound that inhibits the expression or function of at least one gene selected from the group consisting of: Mest, Padi2, Ahr, Klh16, Rasgrp3, Klhi30, Trib2, Rab17, Prkcz, PLCγ2, scin, Pla2g4a, CD7, Il18r1, IL-17a, IL-17f, Sgk3, Gpr15, Pls3 and Zfp187 or the function of a least one gene product encoded by at least one gene selected from the group. In some embodiments compound is selected from a group of compounds consisting of; polyclonal antibodies, or monoclonal antibodies, wherein the antibodies preferentially bind to the cyclosporin-resistant CD8 T-cells. In some embodiments the compound is at least one compound selected from the group consisting of either humanized or non-humanized anti CD7 antibody and humanized or non-humanized anti IL-18 receptor antibody. And in still other embodiments the compound is a siRNA that alters the expression of at least one gene in the cyclosporin resistant CD8 T-cells.

Some embodiments are methods for diagnosing a medical condition, comprising the steps of: analyzing a sample from a patient for the presence of a population of CD8 T-cells that are resistant to levels of cyclosporin and/or rapamycin that inhibits the proliferation of most other subsets of CD8 T-cells. In some embodiments the diagnostic methods include the step of determining if said population of CD8 T-cell in the sample express the IL-18 receptor. In some embodiments the diagnostic method includes: contacting a population of CD8 T-cells with at least one antibody that binds to least one of the following: the IL-18 receptor, and the aryl-hydrocarbon receptor with said CD8 T-cells. In some embodiments CD8 T-cells are cultured in the presence of levels of cyclosporin and or rapamycin that would inhibit the proliferation of most types of CD8 T-cells.

Some embodiments include systems for diagnosing a medical condition; comprising; a sample from a patient, wherein the sample includes CD8 T-cells; a set of conditions for growing the cells in vitro in the presence of levels of cyclosporin and/or rapamycin that inhibit the growth of most types of CD8 T-cells; and an assay for determining if there is a population of CD8 T-cells in the sample that proliferate in the presence of levels of cyclosporin and/or rapamycin that inhibit the growth of most types of CD8 T-cells. In some embodiments the system further includes at least one reagent wherein the reagent binds to an IL-18 receptor on the surface of a subset of CD8 T-cells in the sample or to an aryl hydrocarbon receptor in said subset of CD8 T-cells. In some methods or systems for diagnosing disease sample that includes CD8 T-cells are placed in contact with at least one compound that selectively or at least preferentially interacts with at least one gene product produced by at least one gene selected from the group consisting of: Mest, Padi2, Ahr, Klh16, Rasgrp3, Klhi30, Trib2, Rab17, Prkcz, PLCγ2, scin, Pla2g4a, CD7, Il18r1, IL-17a, IL-17f, Sgk3, Gpr15, Pls3 and Zfp187.

In some embodiments reagents that bind to elements of CD8 T-cells that are resistant to cyclosporin or rapamycin are bound to solid surface such as a bead or a column; this property may be used to help recover and/or identify CD8 T-cells that are resistant to cyclosporin and/or rapamycin. In some embodiments the compounds that interact with CD8 T-cells that are resistant to cyclosporin and/or rapamycin are themselves label with a moiety that can be used to detect them. Such moieties include, but are not limited to, radioisotopes, fluorophores and chemiluminescent groups.

Some embodiments include methods and or systems for diagnosing diseases or conditions in humans or animals that include determining if CD8 T-cells in a sample from the patient expresses at least one gene selected from the group Mest, Padi2, Ahr, Klh16, Rasgrp3, Klhi30, Trib2, Rab17, Prkcz, PLCγ2, scin, Pla2g4a, CD7, Il18r1, IL-17a, IL-17f, Sgk3, Gpr15, Pls3 and Zfp187. In some embodiments the CD8 T-cells are assayed for combination of these genes or gene products. Some embodiments include identifying the CD8-T-cells as being resistant to cyclosporin and/or rapamycin. Some of the assays for gene products may include the use of labelling reagent and techniques such as flow-cytometry. Some of the assays for gene expression may include the use of RT-PCR which may be combined with reverse transcription to quantify the level of mRNA from a given gene.

### Description

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are also within the scope of this disclosure.

Unless specifically stated otherwise the term, ‘therapeutically effective dose,’ as used herein includes an amount of a compound that administered either one time or over the course of treatment cycle affect the health, wellbeing or mortality of a human or animal patient.

Unless specifically stated otherwise the term, ‘semi-professional antigen presenting cells,’-as used herein referrers to non-bone marrow derived cells with low basal levels of MHC class II expression that can be increased with exposure to IFN-gamma (15).

Unless specifically stated otherwise, the term “about” refers to a range of values plus or minus 10% for example about 1.0 refers to it range of values from 0.9 to 1.1.

Cyclosporin may also includes salts of compounds having the formula (E)-14,17,26,32-tetrabutyl-5-ethyl-8-(1-hydroxy-2-methylhex-4-enyl)-1,3,9,12,15,18,20,23,27-nonamethyl-11,29-dipropyl-1,3,6,9,12,15,18,21,24,27,30-undecaazacyclodotriacontan-2,4,7,10,13,16,19,22,25,28,31-undecaone, although other related molecules may also be used to practice some aspects of the invention. The terms, ‘Cyclosporin’ and ‘Cyclosporine’ may both used as neither appears to be the preferred term of art in the literature.

Rapamycin may also includes salts of compounds having the formula (3S,6R,7E,9R,10R,12R,14S,15E,17E,19E,21S,23S, 26R,27R,34aS)-9,10,12,13,14,21,22,23,24,25,26,27,32,33,34,34a-hexadecahydro-9,27-dihydroxy-3-[(1R)-2-[(1S,3R,4R)-4-hydroxy-3methoxycyclohexyl]-1-methylethyl]-10,21-dimethoxy-6,8,12,14,20,26-hexamethyl-23,27-epoxy-3H-pyrido[2,1-c][1,4]-oxaazacyclohentriacontine-1,5,11,28,29 (4H,6H,31H)-pentone.

The term ‘collect’ used in reference to certain cell types includes cell populations that have been enriched in a particular type of cells. Enriched cell populations may include more cells of a particular type of cell in the population than are found in healthy physiological sources.

Chronic allograft rejection is the major cause of organ failure and death in solid organ transplant patients. The pathophysiology driving vascular intimal proliferation and organ fibrosis is not well understood. In cardiac transplantation, interventions focused on atherosclerosis pathogenesis do not prevent cardiac allograft vasculopathy (CAV). Chronic allograft rejection progresses in spite of calcineurin inhibitors or glucocorticoid therapy. Animal models have clearly shown that chronic rejection vasculopathy is T cell dependent and involves both CD4 and CD8 T cell subsets. The unusual histopathology (intimal proliferation and fibrosis instead of tissue necrosis) and resistance to calcineurin blockade, suggests that T cells driving chronic allograft rejection have an immunobiology very different from T cells studied in standard mixed lymphocyte culture systems based on professional antigen presenting cells. Specifically, T cells mediating chronic allograft rejection are likely to function in non-hematopoetic microenvironments, to have reduced cytolytic ability, and to have activation pathways independent of calcineurin/NFAT. Utilizing a novel T cell culture system based on allogeneic epithelial antigen presenting cells (semi-professional APC), a cyclosporin-resistant CD8 T cell clone with minimal cytolytic capability was isolated. Derivation of the novel alloantigen-specific CD8 T cell clones required previous priming with an allogeneic skin graft, implying expansion of this T cell subset during transplant rejection. Characterization and comparison of the cyclosporin-resistant CD8 T cell clone with typical cyclosporin-sensitive CD8 T cells suggests that it is a member of a CD8 T cell subset with a unique cell surface phenotype and novel TCR activation pathways, and that these unique CD8 T cell clones reflect the immunobiology of chronic rejection within nonhematopoetic microenvironments of solid organs and vascular walls.

The scientific literature supports a central role for allogeneic semiprofessional antigen presenting cells as primary targets for chronic allograft rejection, and a role for CD8 T cells as effectors of chronic allograft rejection in the presence of calcineurin inhibitor therapies. Experimentation in murine models has demonstrated that CD8 T cell-mediated chronic rejection includes recognition of MHC class II allo-antigens, and is dependent on production of IFN-γ but not perform or fas ligand killing pathways. Clinical experience and animal models have shown that calcineurin inhibitors do not prevent CAV or BOS. Existing data implicates a noncytolytic IFN-γ-producing CD8 T cell subset with a TCR signaling/activation pathway resistant to calcineurin inhibitors as the mediator of chronic allograft rejection.

Intensive investigations in rodent CAV models have done the most to identify the T cell subsets responsible for mediating chronic allograft rejection. It is clear that CAV is T cell-mediated as allogeneic arterial grafts transplanted into T cell deficient mice do not develop the intimal proliferative lesion characteristic of CAV (16). T cell depletion studies and knockout mice have demonstrated that CD4 T cells play a major role (16-18) and that CD8 T lymphocytes also contribute to the development of CAV (18-20). In a murine model, primed CD8 T cells were sufficient to cause vasculopathy in completely MHC-mismatched aortic grafts. The development of vasculopathy was dependent on T cell IFN-γ, and independent of perform, fas, and fas ligand. In that study, intimal proliferation was also independent of allo-MHC class I expression on the aortic graft, implying CD8 recognition of allo-MHC class II molecules (11). Naïve C57BL/6 CD8 T cells, in nude mice pre-treated with an activating anti-CD40 monoclonal antibody to upregulate the intrinsic co-stimulatory environment, were sufficient to cause CAV in MHC class II-mismatched bm12 cardiac allografts; again implying CD8 recognition of allo-MHC class II molecules as bm12 mice (B6.C-H-2^{bm12}) are isogenic with C57BL/6 mice except for a 3 amino acid change in the bm12 MHC II I-A^{b} beta chain. In the bm12 cardiac allograft study the development of intimal proliferation was also dependent on T cell IFN-γ but not perform or fas ligand (12). CAV histopathology does not resemble the tissue necrosis seen in acute rejection. IFN-γ production, but not potent cytolysis, is required for CD8-mediated CAV.

Various labs have identified a role for CD8 T cells in allograft vasculopathy in models using bm12 model of MHC class II mismatched vascular allografts. At first glance, CD8 recognition of allo-MHC class II molecules would appear to violate a basic cellular immunology paradigm; however T cells participating in rejection can recognize allogeneic donor graft cells via one of two basic mechanisms. ‘Direct allorecognition’ occurs when T cell receptors ‘see’ intact allogeneic major histocompatibility (MHC) molecules on donor cell surfaces; CD4 T cells ‘see’ allo-MHC II, while CD8 T cells ‘see’ allo-MHC I. ‘Indirect allorecognition’ occurs when allogeneic major or minor histocompatibility antigens are taken up by recipient antigen presenting cells (APC) and processed to generate an allopeptide in the context of self-MHC molecules. Peptide fragments of donor MHC class I or class II molecules can be loaded on to recipient self MHC class I or II molecules for presentation to either CD8 or CD4 alloreactive T cells respectively. Direct allorecognition is the predominant mechanism for allorecognition, and generally believed to be the principle mechanism active during acute allograft rejection, while indirect allorecognition has been associated with chronic allograft rejection (reviewed in (21, 22)). The independent research groups that reported CD8-mediated CAV in MHC class II mismatched vascular grafts postulated ‘indirect’ allo-recognition of donor MHC class II molecules. As reported herein CD8 T cells likely directly recognize MHC class II alloantigens in the C57BL/6 versus bm12 transplant model.

The immunobiology of CD8 T cells during solid organ rejection is different than that of CD4 T cells. In patients with acute and chronic graft rejection, CD8 T cells represent a significant portion of the graft-infiltrating-lymphocytes (GIL), and likely participate in graft injury based on their activated state and ability to lyse donor-origin target cells in vitro (23-26). In animal models, CD8 T cells are resistant to costimulation blockade sufficient to induce tolerance in CD4 T cells. For example, blocking CD28 costimulatory pathways with CTLA-4Ig protected intestinal allografts from CD4-mediated but not CD8-mediated rejection (27), and CD8-mediated rejection of allogeneic skin grafts was resistant to combined CD28/CD40 blockade (28). Differential in vivo susceptibility to costimulation blockade implies that CD4 and CD8 T cells utilize different activation pathways. In human transplant patients, chronic allograft rejection and CAV is not prevented by the calcineurin inhibitors cyclosporin A or FK506. In a murine aortic allograft vasculopathy model, CD8 T cells caused allograft vasculopathy in cyclosporin-treated mice. Cyclosporin A prevented vasculopathy caused by CD4 T cells, but was ineffective in preventing vasculopathy caused by CD8 T cells; additionally, the cyclosporin A resistant CD8 T cells had a phenotype that included reduced cytolytic potential (14). Based on those results it is reasonable to conclude that CD8 T cells with a non-cytolytic phenotype, rather than CD4 T cells, appear to have a calcineurin-independent pathway for T cell activation. The literature supports a role for CD8 T cells in cardiac allograft vasculopathy in the setting of MHC class II mismatched grafts. Experimentation in the murine bm12 MHC class II mismatch model has demonstrated that CD8 T cell-mediated CAV is dependent on production of IFN-γ, but not the perform- or fas ligand-mediated killing pathways, and that CD8 lymphocytes are sufficient for the development of chronic rejection (12). The human cardiac transplant experience has shown that calcineurin inhibitors do not prevent CAV. Murine models of CAV show that cyclosporin blocks CD4-mediated but not CD8-mediated CAV, and the murine bm12 model directly implicates CD8 T cells specific for allo-MHC class II in allograft vasculopathy. In aggregate, the existing human and animal model data imply a noncytolytic CD8 T cell subset whose activation pathway remains functional in the presence of calcineurin blockade. Histopathology in clinical specimens and rodent models show a lymphocytic endothelialitis in the vascular intima associated with CAV (29); chronic allograft rejection in other solid organs includes thickening of vascular walls and fibrosis. It is reasonable to postulate that T cells mediating chronic allograft rejection and CAV are being activated in the non-hematopoetic microenvironments. Immunohistochemistry of murine cardiac allografts showed expression of MHC class II molecules on endothelial cells and intimal smooth muscle cells 12 weeks post transplantation, while expression of MHC class II in control cardiac isografts was limited to epicardial macrophages (30). In the mouse bm12 model, CD8 T cell-mediated CAV is postulated to occur via an indirect pathway because of recognition of allo-MHC class II molecules. One hypothesis consistent with this data is that the MHC class II expressed on smooth muscle and endothelial cells serves as alloantigen for direct recognition by CD8 T cells in the pathogenesis of CAV.

As reported herein, during the process of modelling T cell interactions with reproductive tract epithelial cells using the C57BL/6 versus bm12 transplant model, CD8 T cell clones specific for I-A^{bm12} that secreted IFN-γ and a had low cytolytic ability were isolated. One of these CD8 T cell clones, CD8bm12-1, was subsequently used in activation experiments including cyclosporin A, and found to be intrinsically resistant to that drug. That I-A^{bm12}-specific T cell had never been exposed to cyclosporin A in vivo or in vitro prior to those experiments, and is maintained in culture without cyclosporin A with continued resistant to cyclosporin A over 3+ years. This cyclosporin resistant CD8 T cell clone was derived from C57BL/6 mouse that had rejected a bm12 skin graft. T cells from the bm12-skin-graft-primed C57BL/6 mice were cultured ex vivo in a novel nonhematopoetic system using semi-professional bm12-origin oviduct epithelial cells as allogeneic APC. Like post-transplant cardiac allograft endothelial and smooth muscle cells and tracheal epithelium, the oviduct epithelial cell lines used herein express cell surface MHC class II molecules. Without wishing to be restricted to, or limited by, any specific model or hypothesis these results are consistent with a model of chronic allograft rejection in which a cyclosporin-resistant CD8 T cell subset activated by allogeneic epithelial cells (bronchiolitis obliterans syndrome), smooth muscle cells and endothelial cells (chronic allograft vasculopathy) mediates the development of fibrosis and intimal proliferation leading to organ failure. The semi-professional T cell culture system reposted herein, and the unexpected CD8 T cell clones derived using it, provide important insights into the nonhematopoetic immunobiology of solid organ transplant rejection.

Chlamydia trachomatis is an intracellular bacterium that replicates almost exclusively in a single layer of epithelial cells lining the reproductive tract (nonhematopoetic microenvironment), thereby posing a unique problem for the host cellular immune response. As reported herein cloned murine oviduct epithelial cell lines were derived and used them to investigate Chlamydia pathogenesis in the murine model for human Chlamydia trachomatis genital tract infections (31-33). Studies using cloned oviduct epithelial cell lines isolated from B6.C-H2^{bm1}/ByJ and B6.C-H2^{bm12}/KhEg mice have been published (31, 32). Both of these cell lines express low basal levels of MHC class II that can be upregulated with IFN-γ exposure. After successful cloning a C57BL/6 oviduct epithelial cell line designated C57epi.1, it is now possible to assemble a panel of congenic oviduct epithelial cell lines with the following MHC class I (K & D loci) and MHC class II (I-A locus) alleles listed in Table 1.

TABLE 1
MHC Haplotype.

| Mouse strain (cell line) | K | I-A | D |
|---|---|---|---|
| C57BL/6 (C57epi.1) | b | b | b |
| B6.C-H2^{bm1}/ByJ (Bm1.11) | bm1 | b | b |
| B6.C-H2^{bm12}/KhEg (Bm12.4) | b | bm12 | b |

The Bm1.11 and Bm12.4 cell lines are referred to herein as Bm1epi and Bm12epi respectively. These epithelial cell lines were derived in order to study Chlamydia-specific T cells from the reproductive tracts of Chlamydia-immune mice, and to map MHC restriction elements of Chlamydia-specific T cell clones. Before attempting to isolate Chlamydia-specific T cell lines on infected oviduct epithelial APC, an alloantigen was being used to model T cell-epithelial cell interactions.

Briefly, oviduct epithelial cell lines were derived from C57BL/6, B6.C-H2^{bm1}/ByJ and B6.C-H2^{bm12}/KhEg mice to take advantage of the pre-existing transplant rejection model for studying CD4 (bm12 model) versus CD8 (bm1 model) T cell biology. The B6.C-H2^{bm1}/ByJ mouse strain (bm1) is isogenic with the C57BL/6 strain except for a 3 amino acid change in the MHC class I K^{b} allele (34). This small change in the MHC class I K^{b} gene is recognized as foreign by C57BL/6 CD8 T cells, and therefore C57BL/6 mice reject bm1 tissue via an MHC class I-CD8 T cell-mediated mechanism (35, 36). The B6.C-H2^{bm12}/KhEg strain is isogenic with the C57BL/6 strain except for a 3 amino acid change in the MHC class II I-A^{b} beta chain gene (37). This small change in the MHC class 1113 chain is recognized as foreign by C57BL/6 CD4 T cells, and therefore (at least historically) C57BL/6 mice reject bm12 tissue via a CD4 T cell-mediated mechanism (35, 36). One use for this newly isolated oviduct epithelial cell line panel is to examine conventional CD8 T cell-epithelial interactions using the Bm1epi cell line, and unconventional CD4 T cell-epithelial interactions using the Bm12epi cell line. Because the function of epithelial cells as antigen presenting cells is controversial (38), the “alloantigen first” approach reported herein provided the opportunity to develop in vitro protocols for subsequent isolation of Chlamydia-specific CD8 and CD4 T cells on infected epithelial antigen presenting cells.

Using a variant of our published protocol (39), T cell lines and clones were derived from C57BL/6 mice primed with full thickness allogeneic skin grafts using the epithelial cell lines as APC in vitro. Skin, though not vascular in origin, is composed of stroma including blood vessels with an overlying specialized epithelium. Memory T cells from skin-graft-primed mice recovered using allogeneic oviduct epithelial APCs in vitro include, by definition, T cell subsets able to interact with epithelial cells without becoming anergized. T cells recovered using this experimental approach provide a powerful insight into T cell interactions with semi-professional antigen presenting cells such as endothelial and epithelial cells. This experimental model provides an insight into T cell immunobiology operative in the semiprofessional microenvironments of solid organs and vascular walls during development of chronic allograft rejection.

For isolation of bm1-specific CD8 T cell clones, C57BL/6 female mice were tail grafted with B6.C-H2^{bm1}/ByJ full thickness skin grafts that were rejected by day 14 post-transplantation. The mice were rested for eight additional weeks before harvesting splenocytes (memory T cells) and plating them on uninfected monolayers of Bm1epi. To isolate bm12-specific CD4 T cell clones, C57BL/6 female mice were tail grafted with B6.C-H2^{bm12}/KhEg full thickness skin grafts; splenocytes were harvested as above and plated on uninfected monolayers of Bm12epi. To mimic the cytokine milieu at the epithelial interface during genital tract infections, the lymphocyte culture medium included recombinant IL-1, IL-6, IL-7, IL-15, TNF-α, IFN-α and IFN-β at concentrations reflecting levels made by oviduct epithelial cells when infected by C. muridarum (32). IL-1, IL-6, IL-7 and TNF-α are all found in human cardiac allografts undergoing rejection (40). Though IFN-α/β mRNA has not been documented in allograft rejection, human vascular smooth muscle cells treated with TNF-α upregulate IFN-β mRNA (41), and rejecting cardiac allografts have significant IFN-γ (42) which has overlapping biological activities with IFNα/β. IL-15 is constitutively made by stromal cell types and its expression is upregulated in arterial wall injury (43). The culture medium also contained a small amount of IL-2. The T cells recovered in these experimental systems grew out in a cytokine milieu similar to that documented in rejecting cardiac allografts.

Memory T cells recovered from mice primed with bm1 skin grafts vigorously responded to the Bm1epi oviduct epithelial cells in culture. A MHC class I-restricted K^{bm1}-specific CD8 T cell clone designated CD8bm1 was derived by limiting dilution and kept for further studies. CD8bm1 recognizes bm1 bone marrow-derived macrophage cell populations (BMDM) (44), but not syngeneic C57BL/6 BMDM, or C57BL/6 BMDM pulsed/cross-primed with Bm1epi cell membranes (data not shown). These results are consistent with ‘direct’ allorecognition of the K^{bm1} molecule. CD8bm1 makes IFN-gamma, and on that basis it is a conventional alloreactive “Tcl” CD8 T cell clone that recognizes a classic MHC class I alloantigen, K^{bm1}.

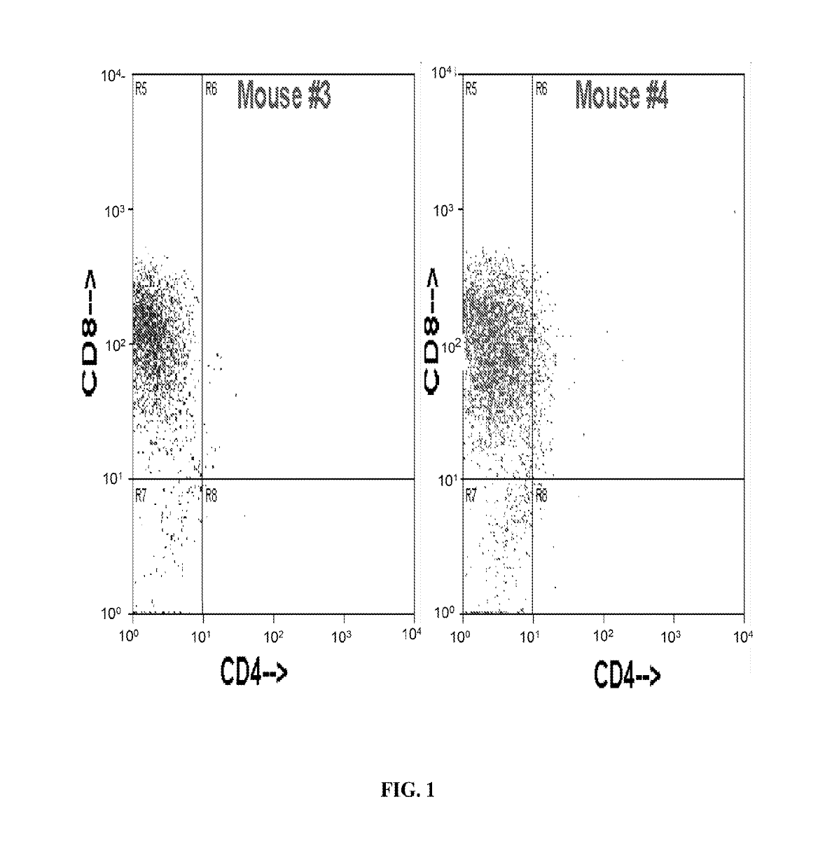

In part, because MHC class II restricted antigen presentation by epithelial cells is both controversial and unexpected, experiments were conducted to determine whether alloreactive CD4+ T cells could be derived on the Bm12epi cell line. Briefly, splenic memory T cells harvested from C57BL/6 mice primed with bm12 skin grafts responded vigorously when plated on the Bm12epi. All wells in this assay had greater than 1000 ρg/ml IFN-γ, epithelial monolayers were lysed, and activated T cells readily recovered on day 5 of the primary cultures. The same bm12-primed C57BL/6 splenocytes did not respond to Bm1epi (H-2K^{bm1}). Additionally, unprimed naïve C57BL/6 mouse splenocytes co-cultured with Bm12epi were not significantly activated, and T cells could not be recovered on day 5 of those primary cultures. IFN-γ production, lysis of the epithelial monolayers and recovery of T cells on culture day 5 were dependent on previous priming with a bm12 allogeneic skin graft. This pattern indicates that the bm12-specific T cell lines recovered in this epithelial-APC-based system originated from a population of T cells expanded during rejection of the bm12 skin grafts. One of the surprising results of the study is that all the bm12-primed/Bm12epi-derived T cell lines, 3 of 3 from three different mice, were >99.5% CD8⁺ T cells rather than the expected CD4⁺ T cells. Two CD8⁺ I-A^{bm12}-specific T cell clones, designated CD8bm12-1 and CD8bm12-2, derived from two different mice were kept for further study. The experiment was repeated with two additional mice, and again cultures yielded exclusively CD8 T cells (FIG. 1). At the same time, consistent with previously published data (35), standard C57BL/6 anti-irradiated bm12 splenocyte mixed lymphocyte reactions (MLR) yielded predominantly CD4⁺ T cells with CD4 to CD8 ratios of ˜10 to 1 (data not shown), suggesting that the immunobiology of solid organ transplant rejection differs significantly from that of standard mixed lymphocyte reactions.

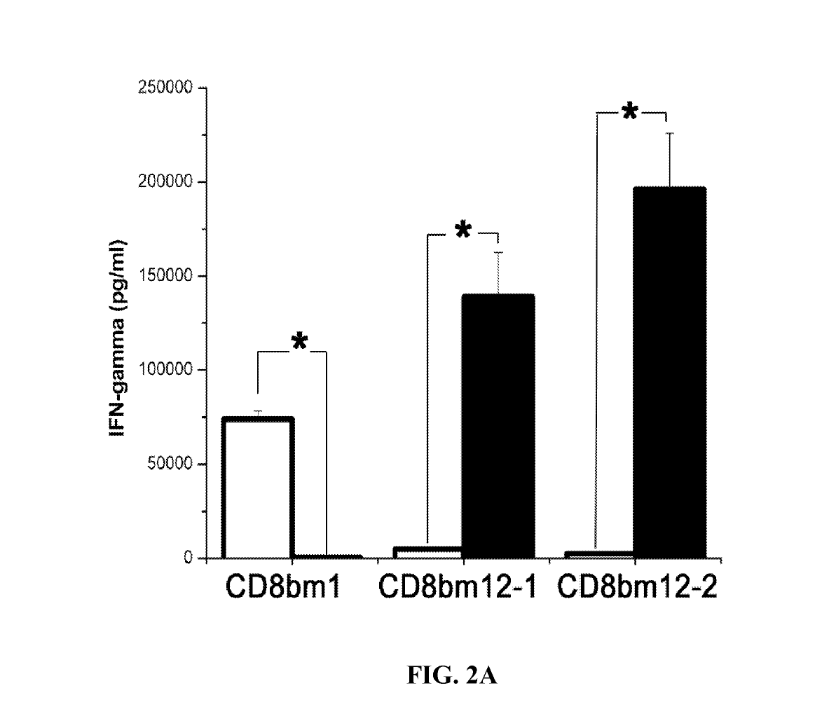

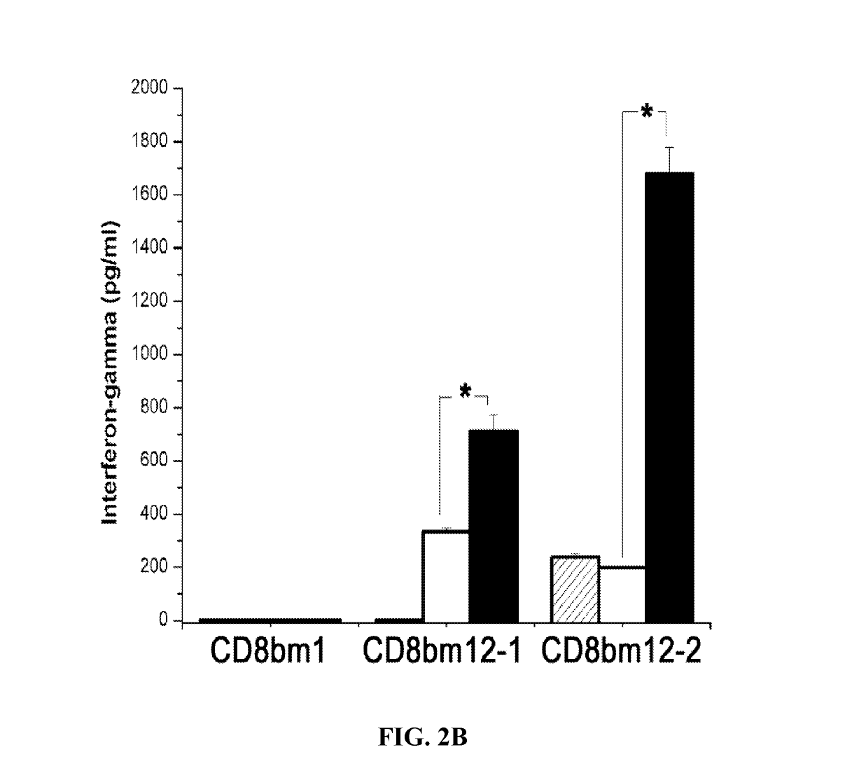

The antigen specificity of the epithelial-derived CD8 T cell clones was measured by activating them with Bm12epi (I-A^{bm12}) and Bm1epi (K^{bm1}) epithelial cells. As illustrated in FIG. 2A, the CD8bm1 T cell clone recognized Bm1epi but not Bm12epi; consistent with recognition of the MHC class I allo-K^{bm1} molecule. Conversely, the CD8bm12-1 and CD8bm12-2 T cell clones recognized Bm12epi but not Bm1epi, consistent with specificity for the MHC class II allo-I-A^{bm12} molecule. The MHC class II specificity of the CD8bm12-1 and CD8bm12-2 T cell clones was further tested by transfecting CL.7 fibroblasts (MHC class II negative, Balb/c H-2^{d} origin) with invariant chain (ATCC# MGC-6517), I-A^{b} alpha chain (ATCC# MGC-30249) and I-A^{bm12} beta chain (cloned and sequenced from Bm12epi), and subsequently isolating a cloned CL.7 cell line expressing cell surface MHC class II I-A^{bm12} by flow cytometry (data not shown) designated CL.7I-A^{bm12}. The CD8bm12-1 and CD8bm12-2 T cell clones, but not the CD8bm1 T cell clone, recognized the CL.7I-A^{bm12} cell line (FIG. 2B). Recognition of I-A^{bm12} in the setting of the non-self MHC class I molecules K^{d} and D^{d} essentially ruled out endogenous cross-priming within the Bm12epi cell line; i.e. CD8bm12-1 and CD8bm12-2 do not recognize I-A^{bm12} peptide fragments loaded onto K^{b} or D^{b} MHC class I molecules. Furthermore, the CD8bm12-1 and CD8bm12-2 T cell clones were not activated by syngeneic C57BL/6 BMDM cross-primed with Bm12epi cell membranes, nor were these clones activated by syngeneic BMDM pulsed with three overlapping 20-mer peptides containing the 3 amino acid change in I-A^{b} that constitutes the I-A^{bm12} allele (data not shown).

The I-A^{bm12} beta chain allele was examined to determine if it had any of the predicted CD8 T cell epitopes on MHC class I K^{b}, D^{b}, K^{d} and D^{d} molecules utilizing the MAPPP prediction algorithm. No bm12-specific peptide fragments were predicted to bind to K^{b}, D^{b}, or D^{d}. The MAPPP algorithm predicted a single I-A^{bm12} specific-peptide (EYWNSQPEFL, SEQ ID No. 1), containing one of the three amino acids (F) that differ between C57BL/6 and bm12 mice, that may bind K^{d}. CL.7 fibroblasts pulsed with EYWNSQPEFL, SEQ ID No. 1 were not recognized by either CD8bm12-1 or CD8bm12-2 (data not shown).

CD8bm1 is a conventional MHC class I-restricted alloreactive CD8 T cell that ‘directly recognizes’ H-2K^{bm1}. CD8bm12-1 and CD8bm12-2 are MHC class II-specific alloreactive CD8 T cells that ‘directly recognize’ the MHC class II I-A^{bm12} molecule. MHC class II-specific CD8 T cells have been isolated from standard MLRs (45, 46), including C57BL/6 versus bm12 (47), virus-infected animals (48, 49) and CD4-deficient transgenic mice expressing a MHC class II-specific TCR transgene (50). Because CD8 T cells were a small minority in standard C57BL/6 versus bm12 mixed lymphocyte reactions (<10%), but the dominant population in these nonhematopoetic allo-system (>99.5%), an explanation consistent with these results is that MHC class II-restricted CD8 T cells may be more common in nonhematopoetic microenvironments of solid organs and the vascular walls. Also, consistent with these results is that the CD8 T cells reported herein to recognize MHC class II I-A^{bm12} are contributing to cardiac allograft vasculopathy demonstrated by Fischbein et al. in the C57BL/6 versus bm12 cardiac allograft model (12, 18, 19) ‘directly’, rather than ‘indirectly.’

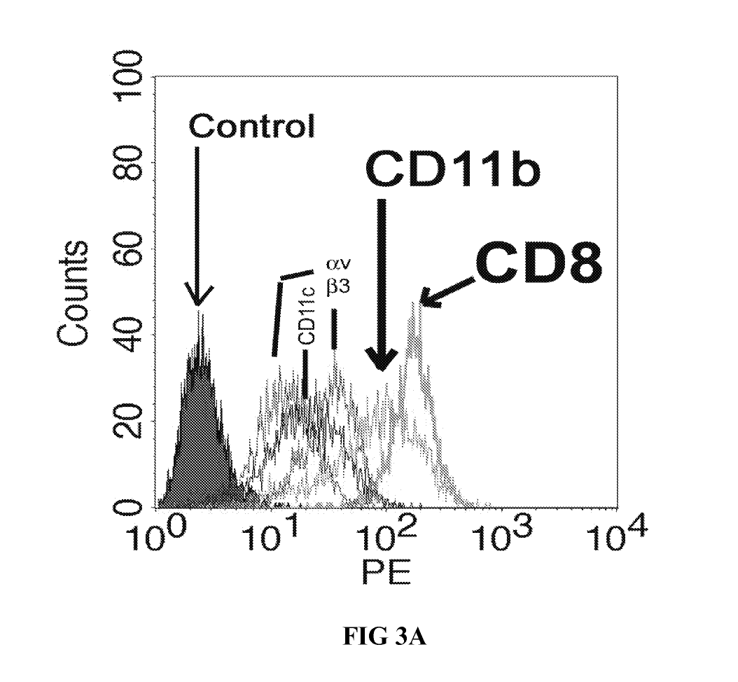

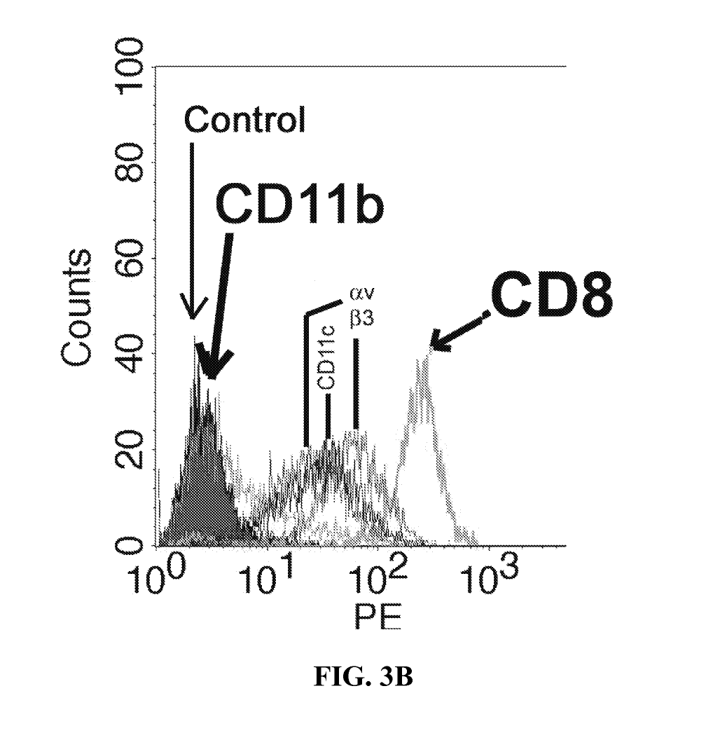

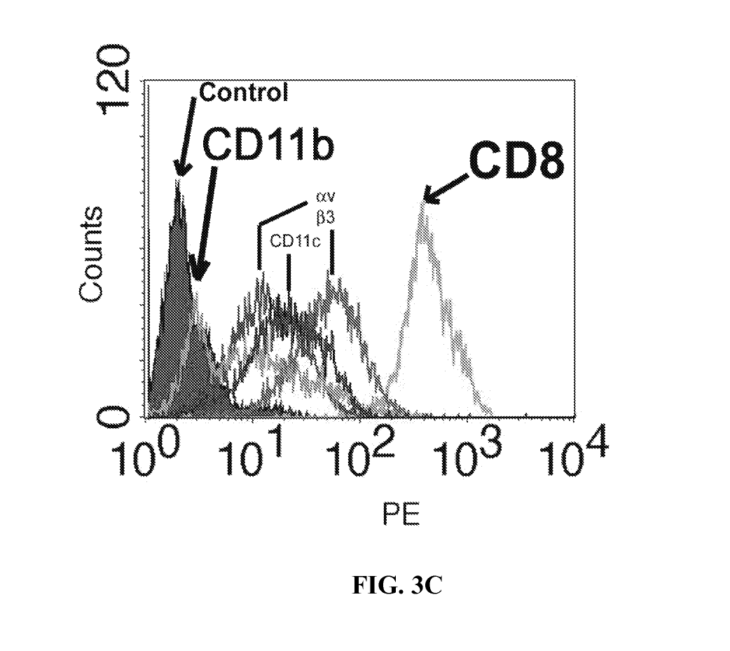

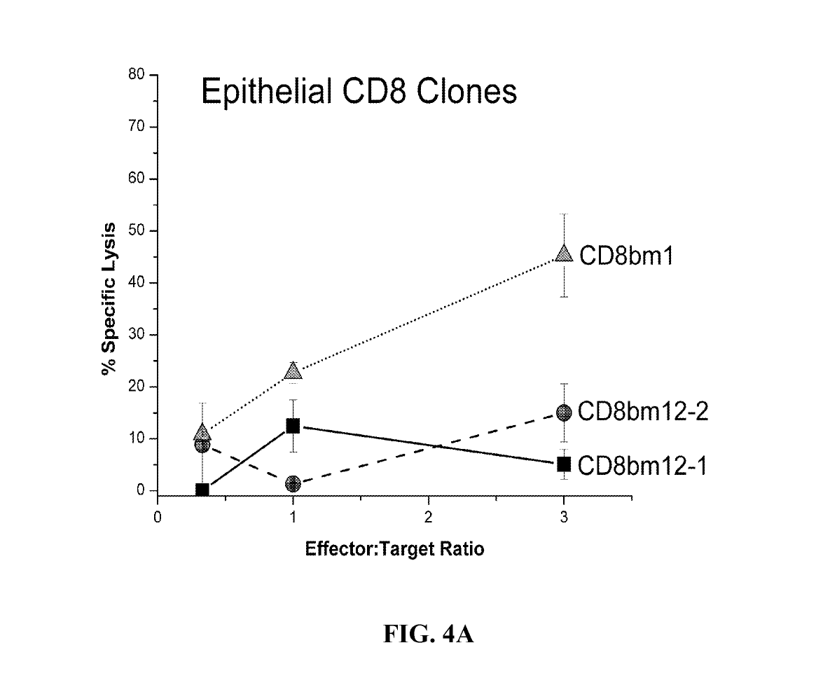

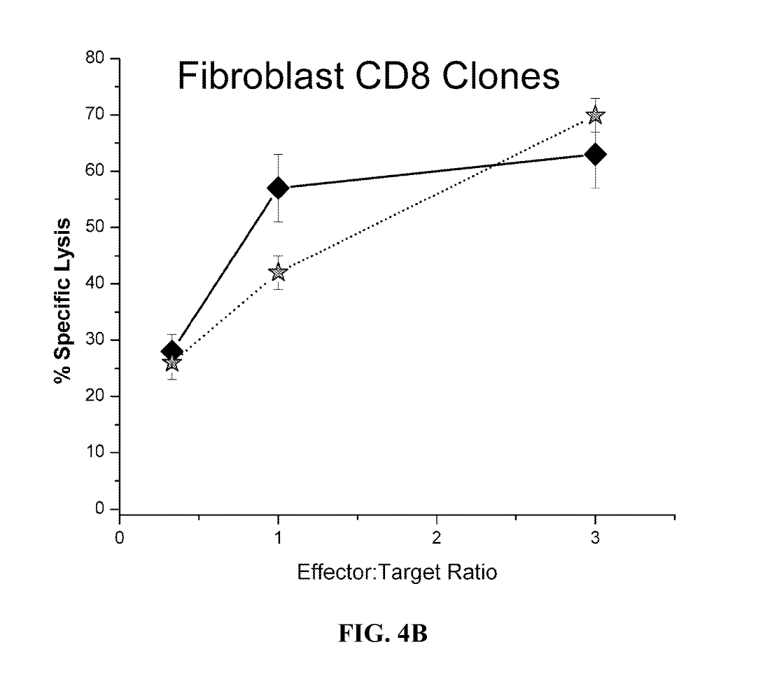

Referring now to FIG. 3, the MHC class I-restricted CD8bm1 (FIG.3a) and MHC class II-restricted CD8bm12-1 (FIG. 3b) and CD8bm12-2 (FIG. 3c) T cell clones are CD8+. Referring now to FIG. 4. The cytolytic function of CD8 T cell clones was measured in four hour assays. The data presented in FIG. 4A were determined with the epithelial-derived CD8 T cell clones CD8bm1, CD8bm12-1 and CDbm12-2 measured against their respective alloepithelial cell line targets Bm1epi and Bm12epi. The data presented in FIG. 4B were collected with two fibroblast-derived alloreactive CD8 T cell clones specific for H-2^{d} measured against CL.7 fibroblast targets. The CD8bm1 T cell clone cytolysis of Bm1.epi cells measured at an effector to target ratio of 3:1 is similar to that of ^{t}he fibroblast-derived T cell clones, while the CD8bm12-1 and CD8bm12-2 clones do not demonstrate meaningful cytolysis of Bm12.epi in short term CTL assays. Both CD8bm12-1 and CD8bm12-2 were significantly less cytolytic than CD8bm1 and fibroblast-derived CD8 T cell clones, a phenotype consistent with cyclosporin-resistant noncytolytic CD8 T cells mediating CAV in mice treated with cyclosporin A (14).

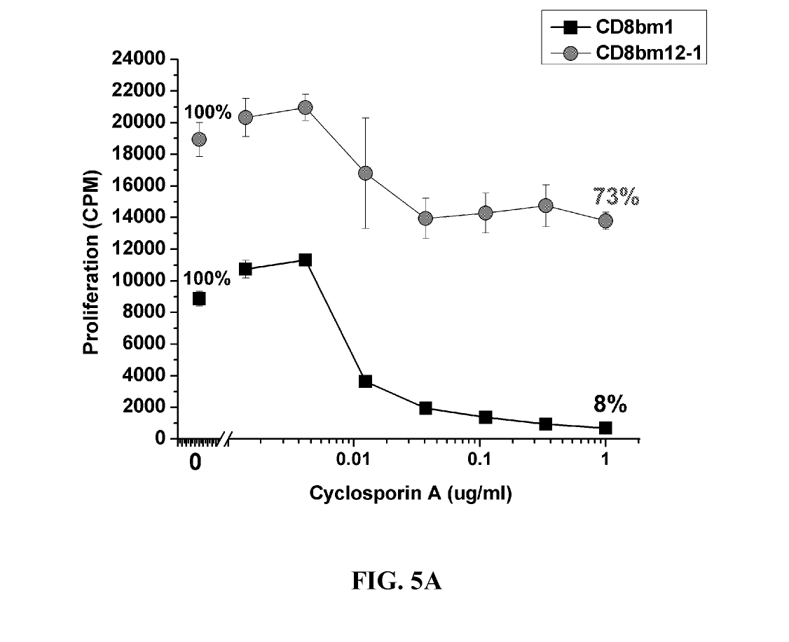

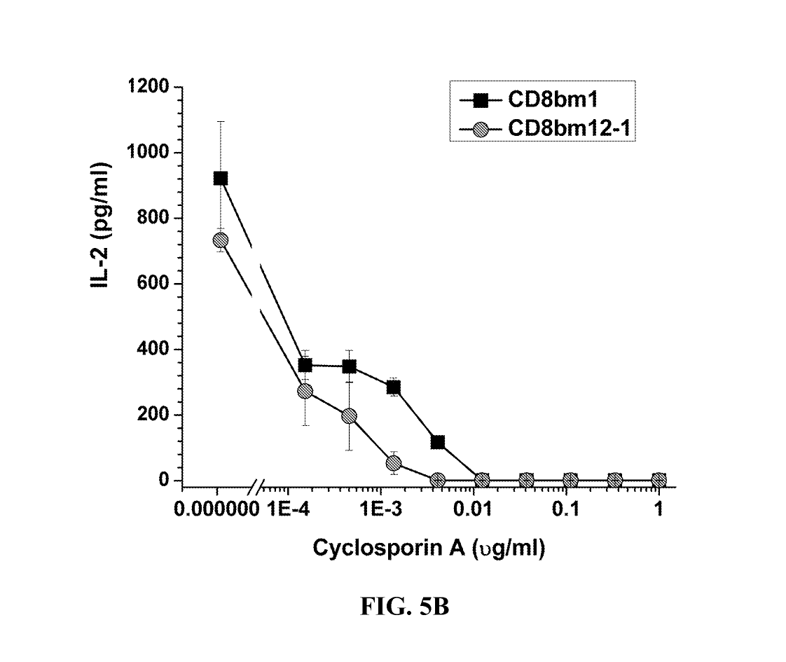

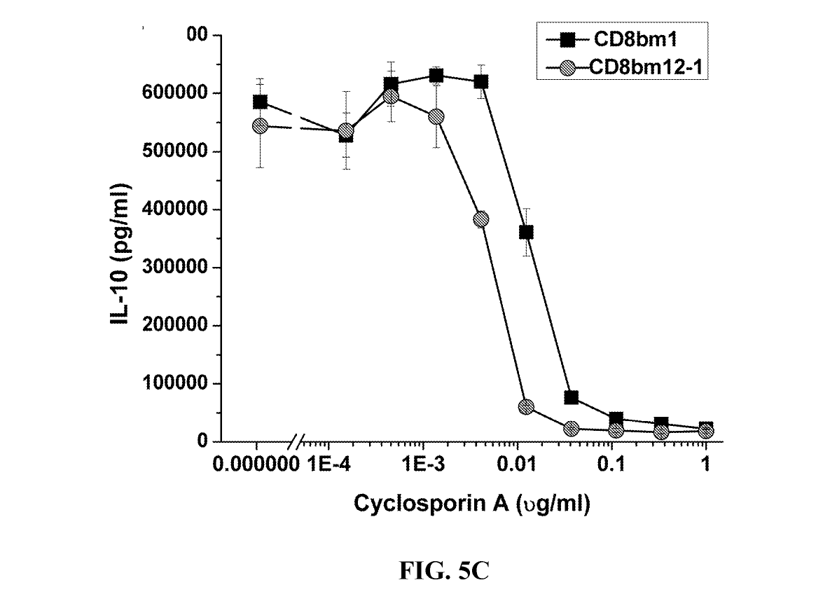

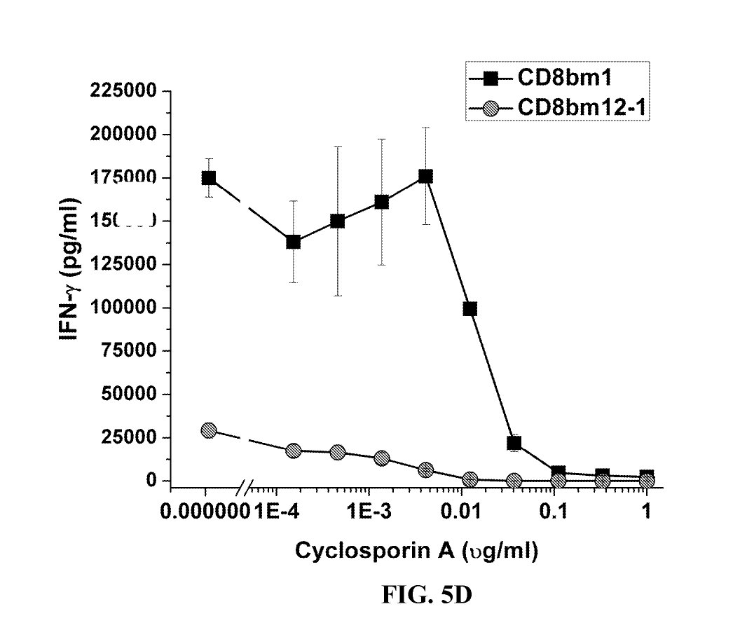

Referring now to FIG. 5a. Further implicating the CD8bm12-1/CD8bm12-2 CD8 T cell type in vasculopathy immunobiology is that CD8bm12-1 cell proliferation to an immobilized anti-CD3 stimulus (T cell receptor complex) is resistant to cyclosporin A, while the conventional CD8bm1 clones proliferation is completely inhibited by cyclosporin A under the same conditions. The CD8bm12-1 clone proliferated as well in the presence of 1 ug/ml of cyclosporin as the CD8bm1 cell did in the absence of the inhibitor. The lower level of proliferation of CD8bm12-1 in the presence of cyclosporin is consistent with the chronicity of chronic allograft rejection (years rather than weeks). Unfortunately in order to conserve reagents and resources the passing of the CD8bm12-2 T cell clone had stopped prior to initiating the cyclosporin experiments. Accordingly, CD8bm12-2 was never tested for cyclosporin resistance. However, CD8bm12-1 is not likely to be an anomalous T cell clone isolated by chance (e.g. a spontaneous mutation in calcineurin that blocks binding of cyclosporin) as CD8bm12-1 is very sensitive to cyclosporin inhibition of IL-2 production (FIG. 5B), IL-10 production (FIG. 5C), and IFN-gamma production (FIG. 5D). As the known mouse and human T cell activation pathways are virtually identical gene for gene, and chronic allograft vasculopathy in mice treated with cyclosporin is histopathologically virtually identical to the clinical disease in humans, the results obtained in the murine experimental model is directly applicable to humans.

Gene expression micro array analysis was performed comparing the novel CD8bm12-1 T cell clone to the conventional “Tc1” CD8bm1 T cell clone using Affymetrix GeneChip® Mouse GENE 1.0 ST obtained from Affymetrix, (Santa Clara, Calif.). In these micro array studies the CD8bm1 and CD8bm12-1 T cell clones were activated by immobilized anti-CD3 antibody in the absence and presence of 1 μg/m lcyclosporin A; 4 repetitions were performed in order to minimize background noise. This activation methodology limits the activation signal to the T cell receptor and downstream TCR signalling pathways as no accessory molecules are engaged under these conditions. Briefly, the preliminary data indicate that the CD8bm12-1 T cell clone represents a novel T cell lineage (Tables 2 & 3). Its unique features include an apparently alternative T cell receptor signalling pathway. For example, CD8bm12-1 has several components of the B cell receptor signalling pathway including critical components PKCzeta (51) and PLCγ2 (52), while having low levels of Lcp2 and Grap2 that are critical components of the TCR signalling pathway (53) in conventional T cells. It is likely that the novel TCR signalling pathways in CD8bm12-1 contribute to its ability to proliferate in the presence of cyclosporin A. In addition, exposure of CD8bm12-1 to cyclosporin A up regulates genes that are not induced in the conventional CD8 T cell clone CD8bm1. Table 2 shows a panel of 8 genes uniquely up regulated by cyclosporin A in anti-CD3 activated CD8bm12-1 cells compared to identically treated CD8bm1 cells. Particularly relevant to cyclosporin resistance are the up regulation of additional B cell receptor signalling pathway components including Klh16 (54) and Rasgrp3 (55), and the aryl hydrocarbon receptor. The aryl hydrocarbon receptor (Ahr) is associated with the Th17 CD4 T cell subset (56), is activated in anti-CD3 stimulated CD8bm12-1 cells based on marked up regulation of Scin, a gene whose expression is regulated by Ahr (57) (Table 3), and further up regulated by cyclosporin A (Table 2). Ahr signalling includes interaction with Rel B. RelB/Ahr complexes bind RelB/p52 promoters for the non-canonical NF-kB pathway (58). Activated CD8bm12-1 cells have IL-17a & IL-17f mRNA and could be labelled “Tc17” cells, but they express mRNA for TGF-β3, and make IFN-gamma, which is inconsistent with assigning a Tc17 lineage. In published studies of Tc17 T cells, those cells have been derived using artificial conditions including Tbet knockout mice (13) and in vitro cytokine manipulation (59). In Tbet knockout mice, the levels of IL-17 mRNA correlated with levels of RORγT, a lineage marker for Th17 T cells, suggesting that Tbet knockout Tc17 cells may actually be Th1 cells artifactually expressing CD8 as a consequence of lacking the critical Th1 transcription factor Tbet. In the cytokine manipulation Tc17 study, cytokine manipulation generated Tc17 T cells that reverted to Tc1 cells in vivo after adoptive transfer. In so far as it can be determine from the existing literature, no one has isolated Tc17 cells from a natural immune response, either to infectious agents, nominal antigens, or rejection of alloantigen. The CD8bm12-1 T cell clone was isolated from wild type C57BL/6 mice that had rejected a bm12 skin graft. CD8bm12-1 is not related to the Tbet knockout mouse Tc17 subset as the micro array did not show significant mRNA for the RORγt, the transcription factor that defines the Th17 lineage and that correlated with IL-17 production by the Tbet knockout mouse Tc17 subset. CD8bm12-1 is not likely related to Tc17 generated in vitro by cytokine manipulation as those cells also were shown to be RORγT positive, and IL-18R negative. In this micro array anti-CD3 activated CD8bm12-1 is IL-18 receptor positive (mean log signal value=8.56; 29.8 fold>than conventional comparator clone CD8bm1) and did not show significant mRNA for RORγT (mean log signal value <4.5 and no different than CD8bm1). The cell surface IL-18R phenotype may be used to establish the existence of the CD8bm12-1 T cell subset as a causative agent in humans with chronic rejection, as the IL-18R can be used to sort out CD8+CD18R+T cells from patients with chronic rejection for in vitro studies (e.g. cytokines, cyclosporin resistance, activation pathways, etc) and gene expression patterns (lineage markers, e.g. RORγT, and novel genes in Tables 2 & 3). CD7 could potentially be similarly be used as a cell surface marker for sorting for the human CD8bm12-1 like CD8 T cell subset. It is possible that CD8 T cells mediating chronic rejection are uniformly CD7⁺, and that the relevant CAV CD8 T cell phenotype is CD8⁺CD7⁺IL-18R⁺. The CD7 finding has the additional feature in that a humanized anti-CD7 murine monoclonal antibody (SDZCHH380) has already been used in a small clinical trial to prevent acute renal graft rejection, and was shown to be safe and effective compared with anti-OKT3 (60). The humanized anti-CD7 monoclonal antibody, or its logical descendants, may be an ideal therapy for chronic allograft vasculopathy and chronic rejection of other solid organs.

TABLE 2
Cyclosporin-specific changes induced in CD8bm12-1 T cells
Genes up regulated in anti-CD3 activated CD8bm12-1 T cells by CSA:
CD8bm12-1/CSA vs CD8bm1/CSA (Condition 1 = C1)- identifies genes
unique to CSA treated CD8bm12-1 cells
CD8bm12-1/CSA vs CD8bm12-1 (Condition 2 = C2)- quantifies level
of gene induction by CSA

| | C1- | C2- | |
|---|---|---|---|
| Gene | fold * | fold † | Function |
| Mest | 65.8 | 10.1 | ? fxn- upregulated in adipose tissue |
| | | | expansion |
| Padi2 | 29.8 | 6.48 | Peptidyl arginine deiminase, type II- |
| | | | post-translational citrullination |
| | | | of proteins |
| Ahr | 34.95 | 2.88 | Aryl hydrocarbon receptor- its downstream |
| | | | signaling is known to be amplified by |
| | | | cyclosporin A. |
| Klhl6 | 14.35 | 3.01 | Kelch-like 6- involved in B cell receptor |
| | | | (BCR) signaling |
| Rasgrp3 | 16.4 | 5.67 | BCR signaling downstream of PKCzeta |
| Klhl30 | 8.89 | 9.32 | unknown- protein-protein interaction domain |
| Trib2 | 7.78 | 4.42 | Tribbles homolog 2- expression = a growth |
| | | | Advantage ex vivo |
| Rab17 | 5.5 | 5.46 | unknown- Ras family member |

* Welch T-test (log signal) pvalue < 1 × 10\hat{ }−9; Welch T-test (log signal) pvalue < 1 × 10\hat{ }−7.

Reportedly, IL-2 secretion in T cells is dependent on PKC isoform θ (61, 62). It is unlikely that IL-2 secretion by CD8bm12-1 explains its cyclosporin-resistant phenotype as IL-2 secretion by CD8bm1 and CD8bm12-1 was dramatically inhibited by cyclosporin A (FIG. 5b). It is reasonable to postulate that PKCζ, PLCγ2, Rasgrp3, and Klh16 participate in an alternative TCR signalling pathway that becomes especially evident in CD8bm12-1 T cells when the major IL-2-driven production/proliferation pathway through calcineurin is blocked by cyclosporin A. PKC ζ is a major component of B cell receptor signalling, however, interestingly, PCK ζ deficient mice also have deficiencies in T cell activation (51). The genes identified above and detailed in Tables 2 & 3 are all potentially therapeutic targets for mitigating chronic graft rejection. The aryl hydrocarbon receptor provides an opportunity to mitigate chronic rejection caused by the CD8bm12-1 T cell subset as the micro array shows that the Ahr has been activated, based on marked up regulation of Scin, in anti-CD3 activated CD8bm12-1 T cells, with or without exposure to cyclosporin (table 3). In Th17 cells, Ahr maintains a basal level of activation, presumably by endogenous ligands, which can be manipulated to cause heighten levels of Th17 activation or Th17 suppression by synthetic ligands (68). An overall hypothesis consistent with these results is that CD8bm12-1 T cells in the presence of cyclosporin A utilize a B cell-like calcineurin-independent activation pathway. B cells have a cyclosporin-resistant proliferation pathway (63). CD8bm12-1 may be the only T cell clone ever described that can proliferate in the presence of 1 μg/ml of cyclosporin A. The initial characterization provides insight into novel T cell biology that can be exploited to treat chronic allograft vasculopathy and other forms of chronic allograft rejection.

Referring now to Table 3. Important genes up & down regulated in anti-CD3 activated CD8bm12-1 vs anti-CD3 activated CD8bm1 (Condition 3=C3), but not affected in CD3-activated CD8bm12-1 cells by addition of CSA (Condition 2=C2 same as above): i.e., gene expression that differs at baseline in the activated conventional (CD8bm1) and unconventional (CD8bm12-1) CD8 T cell clones.

TABLE 3
Baseline differences between activated
CD8bm12-1 & CD8bm1 T cells.

| | | | |
|---|---|---|---|
| | C3- | C2- | |
| Gene | fold‡ | fold | Function |

B cell signaling

| | | | |
|---|---|---|---|
| Prkcz | 8.38 | 1.10 | PKCzeta- critical component of B cell |
| | | | receptor (BCR) signaling |
| PLCγ2 | 8.06 | −1.14 | Phospholipase C gamma 2- critical |
| | | | component of BCR signaling |
| Gpr183 | 7.64 | 1.46 | B cell migration |

T cell signaling

| | | | |
|---|---|---|---|
| Lcp2 | −84.47 | −2.26 | SLP-76-critical for conventional T cell |
| | | | receptor (TCR) signaling |
| Grap2 | −6.57 | 3.51 | LAT to SLP-76 adaptor |
| Dgkα | −16.34 | 1.14 | Diacyl glyceride kinase α- up regulated |
| | | | in anergic states |
| Lilrb4 | −18.55 | −2.27 | Leukocyte immunoglobulin-like receptor, |
| | | | subfamily B, member 4- a co-inhibitory |
| | | | receptor |

Aryl hydrocarbon receptor

| | | | |
|---|---|---|---|
| Scin | 193.18 | 1.19 | Scinderin- up regulated by activation of aryl |
| | | | hydrocarbon receptor (Ahr) |
| Pla2g4a | 57.32 | 1.38 | Phospholipase A2, group IV- signal |
| | | | transducer for Ahr |

Cell surface markers & cytokines

| | | | |
|---|---|---|---|
| CD7 | 28.47 | −1.12 | CD7 |
| II18r1 | 10.91 | −2.71 | IL-18 receptor |
| IL-17a | 8.5 | −11.1 | IL-17a |
| IL-17f | 7.03 | −8.72 | IL-17f |
| Tgfb3 | 12.28 | 1.3 | TGF-beta 3 |

Other

| | | | |
|---|---|---|---|
| Sgk3 | 26.57 | −1.01 | downstream of PI-3 kinase (survival) |
| Gpr15 | 20.21 | 1.45 | G protein-coupled receptor 15 |
| Pls3 | 6.80 | 1.22 | unknown signaling molecule |
| Zfp187 | 9.80 | 1.03 | unknown transcription factor |

‡Welch T-test (log signal) pvalue < 1 × 10\hat{ }−7.

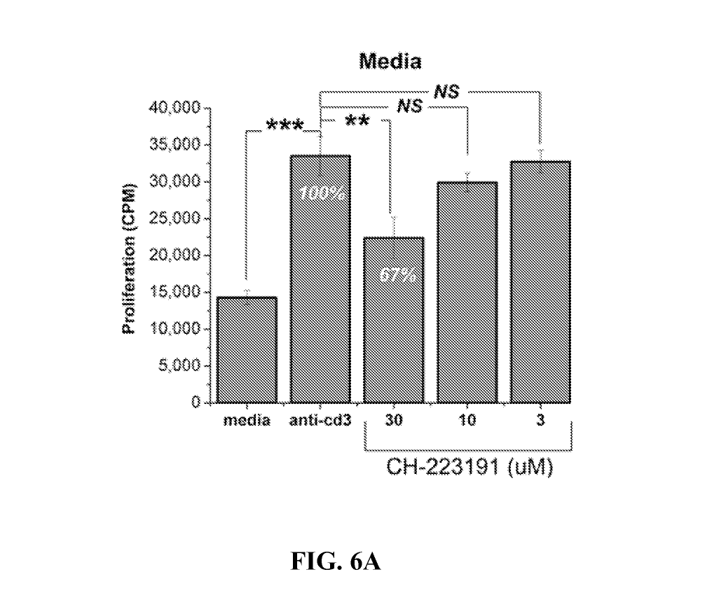

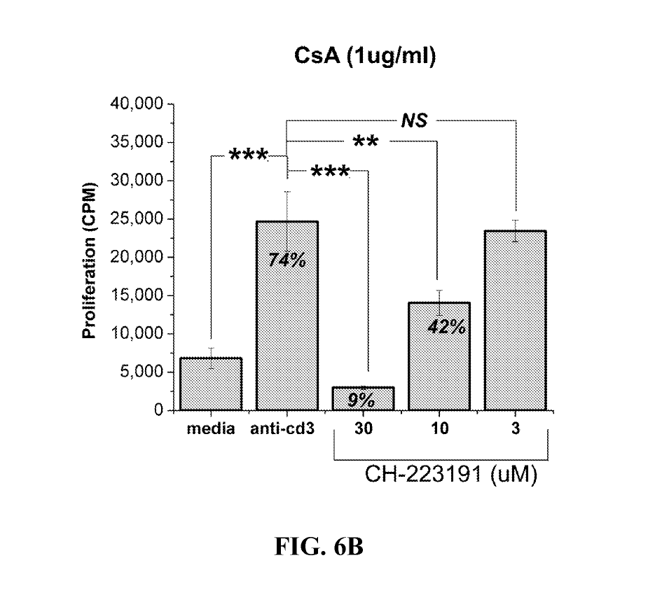

Referring now to FIG. 6A, experiments activating CD8bm12-1 with immobilized anti-CD3 were repeated in the presence of varying concentrations of CH-223191, a compound that antagonizes binding and activation of the aryl hydrocarbon receptor (Ahr). Proliferation of CD8bm12-1 activated by anti-CD3 in the presence of 30 uM CH-223191 was inhibited by 33%; i.e. was only able to proliferate to 67% the level seen when CD8bm12-1 was activated by anti-CD3 in media. FIG. 6A demonstrates that activation of the Ahr is an important event in proliferation of CD8bm12-1 to an anti-CD3 (T cell receptor complex) stimulus. Now referring to FIG. 6B, in the same experiments as shown in FIG. 6A, CD8bm12-1 was activated with immobilized anti-CD3 in media containing 1 μg/ml cyclosporin A in the presence of varying concentrations of CH-223191. In the presence of cyclosporin A CD8bm12-1 cells retained 74% of their proliferation to an anti-CD3 stimulus compared to an anti-CD3 stimulus in regular media. In media containing cyclosporin A presence of 30 μM of the hydrocarbon receptor antagonist CH-223191 reduced CD8bml-12 proliferation to only 9% of that to an anti-CD3 stimulus in regular media; a level of proliferation less than CD8bm12-1 in media without immobilized anti-CD3 (unactivated cells). The data in FIG. 6B demonstrate that aryl hydrocarbon receptor activation is critical to the cyclosporin resistance phenotype of CD8bm12-1, and by extrapolation, that chronic allograft rejection could be treated with cyclosporin A plus an antagonist of the Ahr. All of the results presented in FIGS. 6A and 6B are aggregates of data collected from two independent experiments; the measurement of cell proliferation was at 38-48 hours by pulsing with ³H thymidine. Student t-test value for the data sets were **=pvalue<0.005; ***=pvalue<0.005. All of the percentages in these two figures are comparisons to the proliferation of CD8bm12-1 cells activated with immobilized anti-CD3 in media that did not include inhibitors.

The data presented in FIGS. 6A and 6B, are consistent with the conclusion that that activation of the Aryl Hydrocarbon Receptor (AHR) by an endogenous ligand is critical to the intrinsic resistance of CD8bm12-1 to cyclosporin A.

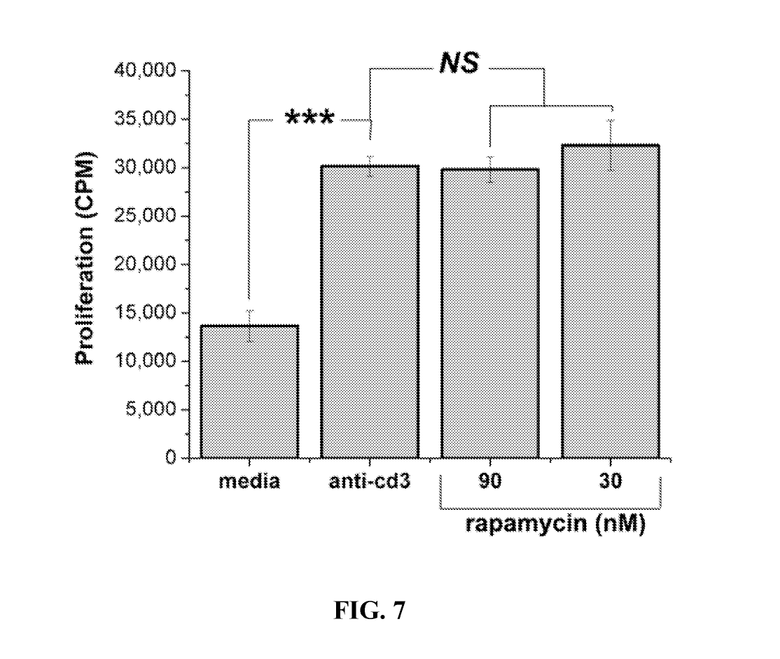

Referring now to FIG. 7, cell proliferation, expressed in counts of ³H thymidine, was measured for CD8bm12-1 cells exposed to media only (unactivated cells), and to media plus immobilized anti-CD-3 antibody (activated CD8bm12-1 cells) in the presence of either, 0, 30 or 90 nM rapamycin. Rapamycin had no significant effect on proliferation of CD8bm12-1 to an anti-CD3 stimulus. These data demonstrate that CD8bm12-1 proliferation to an anti-CD3 stimulus is largely IL-2-independent as rapamycin blocks signalling downstream of the IL-2 receptor. That conclusion is further supported by data shown in FIG. 5A & 5B showing the CD8bm12-1 is able to proliferate to an anti-CD3 stimulus in the presence of cyclosporin A concentrations that drive IL-2 production to undetectable levels. The measurement of cell proliferation was at 38-48 hours by pulsing with ³H thymidine. Students t-test pvalues for the data were <0.0005. These data indicate that activated CD8bm12-1 cells are rapamycin resistant.

The MHC class II-specific CD8 T cell clone CD8bm12-1 reported here is intrinsically resistant to cyclosporin and rapamycin, lacks potent cytolytic activity, expresses the aryl hydro-carbon receptor and may be representative of a cyclosporin-resistant subset of CD8 T cells previously described as critical participants in murine models of cardiac allograft vasculopathy. The identifying materials and methods disclosed here are useful for candidate ‘CAV’ and chronic allograft rejection T cell phenotypes, and activation pathways operative in the immunopathology of cardiac allograft vasculopathy and chronic allograft rejection. These materials and methods can be used to identify new therapeutic targets for treating allograft vasculopathy, and chronic T cell-mediated rejection in other solid organ transplants.

Aspects of the invention include methods for screening, selecting and identifying molecules that can inhibit, reduce or eliminate a subset of CD8 T-cells that contribute to allograft rejections. These methods are amenable to use in both human and animals. For example, all the candidate target molecules for selectively depleting or inhibiting cyclosporin- resistant CD8 T cells (shown in tables 2 & 3) have conserved human homologs. Representative methods for treating allograft rejection disease or for methods for selecting compounds that have the ability or potential to treat allograft rejection disease include the steps of depleting cyclosporin-resistant CD8 T cells using a pre-existing humanized anti-CD7 antibody (60), or new anti-CD7 or anti-IL-18 receptor (IL-18r1) antibody reagents developed for this or other purpose.

The cyclosporin-resistant CD8 T cell subset can predictably be purified from normal individuals, but likely these cells are more readily harvested from transplant patients on calcineurin inhibitor therapies with chronic allograft rejection, by, for example, sorting on the IL-18r1⁺CD8⁺ T cell subset. Purification of this T cell subset can be accomplished using any means known in the art including for example, but not limited to, magnetic bead separation or cell-sorting with a flow cytometer to provide a convenient source of this novel cell type for further biochemical/molecular biology characterization, and for bioassays to identify potential inhibitors of the cyclosporin resistant proliferation pathway.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

### REFERENCES

1. Lodhi, S. A., K. E. Lamb, and H. U. Meier-Kriesche. Solid Organ Allograft Survival Improvement in the United States: The Long-Term Does Not Mirror the Dramatic Short-Term Success. Am J Transplant.
2. Sundaresan, S., E. P. Trulock, T. Mohanakumar, J. D. Cooper, and G. A. Patterson. 1995. Prevalence and outcome of bronchiolitis obliterans syndrome after lung transplantation. Washington University Lung Transplant Group. Ann Thorac Surg 60:1341-1346; discussion 1346-1347.
3. Taylor, D. O., L. B. Edwards, M. M. Boucek, E. P. Trulock, M. C. Deng, B. M. Keck, and M. I. Hertz. 2005. Registry of the International Society for Heart and Lung Transplantation: twenty-second official adult heart transplant report--2005. J Heart Lung Transplant 24:945-955.
4. Norman, B. C., D. A. Jacobsohn, K. M. Williams, B. K. Au, M. A. Au, S. J. Lee, C. K. Moravec, and J. W. Chien. Fluticasone, azithromycin and montelukast therapy in reducing corticosteroid exposure in bronchiolitis obliterans syndrome after allogeneic hematopoietic SCT: a case series of eight patients. Bone Marrow Transplant.
5. McDyer, J. F. 2007. Human and murine obliterative bronchiolitis in transplant. Proc Am Thorac Soc 4:37-43.
6. Panoskaltsis-Mortari, A., K. V. Tram, A. P. Price, C. H. Wendt, and B. R. Blazar. 2007. A new murine model for bronchiolitis obliterans post-bone marrow transplant. Am J Respir Crit Care Med 176:713-723.
7. Fernandez, F. G., A. Jaramillo, C. Chen, D. Z. Liu, T. Tung, G. A. Patterson, and T. Mohanakumar. 2004. Airway epithelium is the primary target of allograft rejection in murine obliterative airway disease. Am J Transplant 4:319-325.
8. Jungraithmayr, W., P. Vogt, I. Inci, S. Hillinger, S. Arni, S. Korom, and W. Weder. A model of chronic lung allograft rejection in the rat. Eur Respir J 35:1354-1363.
9. Wasowska, B., K. J. Wieder, W. W. Hancock, X. X. Zheng, B. Berse, J. Binder, T. B. Strom, and J. W. Kupiec-Weglinski. 1996. Cytokine and alloantibody networks in long term cardiac allografts in rat recipients treated with rapamycin. J Immunol 156:395-404.
10. Servais, A., V. Meas-Yedid, O. Toupance, Y. Lebranchu, A. Thierry, B. Moulin, I. Etienne, C. Presne, L. B. Hurault de, P. Le Pogamp, Y. Le Meur, D. Glotz, C. Hayem, J. C. Olivo Marin, and E. Thervet. 2009. Interstitial fibrosis quantification in renal transplant recipients randomized to continue cyclosporin or convert to sirolimus. Am J Transplant 9:2552-2560.
11. Skaro, A. I., R. S. Liwski, J. Zhou, E. L. Vessie, T. D. Lee, and G. M. Hirsch. 2005. CD8+ T cells mediate aortic allograft vasculopathy by direct killing and an interferon-gamma-dependent indirect pathway. Cardiovasc Res 65:283-291.
12. Schnickel, G. T., D. Whiting, G. R. Hsieh, J. J. Yun, M. P. Fischbein, M. C. Fishbein, W. Yao, A. Shfizadeh, and A. Ardehali. 2004. CD8 lymphocytes are sufficient for the development of chronic rejection. Transplantation 78:1634-1639.
13. Burrell, B. E., K. Csencsits, G. Lu, S. Grabauskiene, and D. K. Bishop. 2008. CD8+ Th17 mediate costimulation blockade-resistant allograft rejection in T-bet-deficient mice. J Immunol 181:3906-3914.
14. Vessie, E. L., G. M. Hirsch, and T. D. Lee. 2005. Aortic allograft vasculopathy is mediated by CD8(+) T cells in Cyclosporin A immunosuppressed mice. Transpl Immunol 15:35-44.
15. Kosaka, H., C. D. Surh, and J. Sprent. 1992. Stimulation of mature unprimed CD8+ T cells by semiprofessional antigen-presenting cells in vivo. J Exp Med 176:1291-1302.
16. Shi, C., W. S. Lee, Q. He, D. Zhang, D. L. Fletcher, Jr., J. B. Newell, and E. Haber. 1996.

Immunologic basis of transplant-associated arteriosclerosis. Proc Natl Acad Sci USA 93:4051-4056.
17. Szeto, W. Y., A. M. Krasinskas, D. Kreisel, A. S. Krupnick, S. H. Popma, and B. R. Rosengard. 2002. Depletion of recipient CD4+ but not CD8+ T lymphocytes prevents the development of cardiac allograft vasculopathy. Transplantation 73:1116-1122.
18. Fischbein, M. P., J. Yun, H. Laks, Y. Irie, M. C. Fishbein, B. Bonavida, and A. Ardehali. 2002. Role of CD8+lymphocytes in chronic rejection of transplanted hearts. J Thorac Cardiovasc Surg 123:803-809.
19. Fischbein, M. P., J. Yun, H. Laks, Y. Irie, M. C. Fishbein, M. Espejo, B. Bonavida, and A. Ardehali. 2001. CD8+ lymphocytes augment chronic rejection in a MHC class II mismatched model. Transplantation 71:1146-1153.
20. Raisky, O., B. M. Spriewald, K. J. Morrison, S. Ensminger, T. Mohieddine, J. F. Obadia, M. H. Yacoub, and M. L. Rose. 2003. CD8(+) T cells induce graft vascular occlusion in a CD40 knockout donor/recipient combination. J Heart Lung Transplant 22:177-183.
21. Womer, K. L., M. H. Sayegh, and H. Auchincloss, Jr. 2001. Involvement of the direct and indirect pathways of allorecognition in tolerance induction. Philos Trans R Soc Lond B Biol Sci 356:639-647.
22. Rogers, N. J., and R. I. Lechler. 2001. Allorecognition. Am J Transplant 1:97-102.
23. Poindexter, N., S. Shenoy, T. Howard, M. W. Flye, and T. Mohanakumar. 1997. Allograft infiltrating cytotoxic T lymphocytes recognize kidney-specific human minor histocompatibility antigens. Clin Transplant 11:174-177.
24. Ouwehand, A. J., C. C. Baan, L. M. Vaessen, N. H. Jutte, A. H. Balk, E. Bos, F. H. Claas, and W. Weimar. 1992. The influence of HLA-mismatches on phenotypic and functional characteristics of graft infiltrating lymphocytes after heart transplantation. Transpl Int 5 Suppl 1:S673-675.
25. Jutte, N. H., M. H. van Batenburg, C. R. Daane, P. Heijse, L. M. Vaessen, A. H. Balk, B. Mochtar, F. H. Claas, and W. Weimar. 1992. Lysis of heart endothelial cells from donor origin by cardiac graft infiltrating cells. Transpl Int 5 Suppl 1:S645-647.
26. Hadley, G. A., C. Charandee, M. R. Weir, D. Wang, S. T. Bartlett, and C. B. Drachenberg. 2001. CD103+ CTL accumulate within the graft epithelium during clinical renal allograft rejection. Transplantation 72:1548-1555.
27. Newell, K. A., G. He, Z. Guo, O. Kim, G. L. Szot, I. Rulifson, P. Zhou, J. Hart, J. R. Thistlethwaite, and J. A. Bluestone. 1999. Cutting edge: blockade of the CD28/B7 costimulatory pathway inhibits intestinal allograft rejection mediated by CD4+ but not CD8+ T cells. Jlmmunol 163:2358-2362.
28. Trambley, J., A. W. Bingaman, A. Lin, E. T. Elwood, S. Y. Waitze, J. Ha, M. M. Durham, M. Corbascio, S. R. Cowan, T. C. Pearson, and C. P. Larsen. 1999. Asialo GM1(+) CD8(+) T cells play a critical role in costimulation blockade-resistant allograft rejection. J Clin Invest 104:1715-1722.
29. Paavonen, T., A. Mennander, I. Lautenschlager, S. Mattila, and P. Hayry. 1993. Endothelialitis and accelerated arteriosclerosis in human heart transplant coronaries. J Heart Lung Transplant 12:117-122.
30. Hasegawa, S., G. Becker, H. Nagano, P. Libby, and R. N. Mitchell. 1998. Pattern of graft- and host-specific MHC class II expression in long-term murine cardiac allografts: origin of inflammatory and vascular wall cells. Am J Pathol 153:69-79.
31. Derbigny, W. A., M. S. Kerr, and R. M. Johnson. 2005. Pattern recognition molecules activated by Chlamydia muridarum infection of cloned murine oviduct epithelial cell lines. J Immunol 175:6065-6075.
32. Johnson, R. M. 2004. Murine oviduct epithelial cell cytokine responses to Chlamydia muridarum infection include interleukin-12-p70 secretion. Infect Immun 72:3951-3960.
33. Nelson, D. E., D. P. Virok, H. Wood, C. Roshick, R. M. Johnson, W. M. Whitmire, D. D. Crane, O. Steele-Mortimer, L. Kari, G. McClarty, and H. D. Caldwell. 2005. Chlamydial IFN-{gamma} immune evasion is linked to host infection tropism. Proc Natl Acad Sci USA 102:10658-10663.
34. Schulze, D. H., L. R. Pease, S. S. Geier, A. A. Reyes, L. A. Sarmiento, R. B. Wallace, and S. G. Nathenson. 1983. Comparison of the cloned H-2Kbm1 variant gene with the H-2Kb gene shows a cluster of seven nucleotide differences. Proc Natl Acad Sci USA 80:2007-2011.
35. Sprent, J., M. Schaefer, D. Lo, and R. Korngold. 1986. Properties of purified T cell subsets. II. In vivo responses to class I vs. class II H-2 differences. J Exp Med 163:998-1011.
36. Vallera, D. A., P. A. Taylor, J. Sprent, and B. R. Blazar. 1994. The role of host T cell subsets in bone marrow rejection directed to isolated major histocompatibility complex class I versus class II differences of bm1 and bm12 mutant mice. Transplantation 57:249-256.
37. McIntyre, K. R., and J. G. Seidman. 1984. Nucleotide sequence of mutant I-A beta bm12 gene is evidence for genetic exchange between mouse immune response genes. Nature 308:551-553.
38. Marelli-Berg, F. M., and R. I. Lechler. 1999. Antigen presentation by parenchymal cells: a route to peripheral tolerance? Immunol Rev 172:297-314.
39. Johnson, R., D. W. Lancki, and F. W. Fitch. 1993. Accessory molecules involved in antigen-mediated cytolysis and lymphokine production by cytotoxic T lymphocyte subsets. I. Identification of functions for the T cell surface molecules Ly-6C and Thy-1. Journal of Immunology 151:2986-2999.
40. Salom, R. N., J. A. Maguire, and W. W. Hancock. 1998. Endothelial activation and cytokine expression in human acute cardiac allograft rejection. Pathology 30:24-29.
41. Palmer, H., and P. Libby. 1992. Interferon-beta. A potential autocrine regulator of human vascular smooth muscle cell growth. Lab Invest 66:715-721.
42. Christopher, K., Y. Liang, T. F. Mueller, R. DeFina, H. He, K. J. Haley, M. A. Exley, P. W. Finn, and D. L. Perkins 2004. Analysis of the major histocompatibility complex in graft rejection revisited by gene expression profiles. Transplantation 78:788-798.
43. Cercek, M., M. Matsumoto, H. Li, K. Y. Chyu, A. Peter, P. K. Shah, and P. C. Dimayuga. 2006. Autocrine role of vascular IL-15 in intimal thickening. Biochem Biophys Res Commun 339:618-623.
44. Falk, F. A. 2001. Isolation of bone marrow-derived macrophages. In Current Protocols In Immunology. J. E. Coligan, A. M. Kruisbeek, D. H. Marguiles, E. M. Shevach, and W. Strober, eds. John Wiley & Sons Inc., Bethesda, Md. 14.11.13-14.11.19.
45. Suzuki, H., K. Eshima, Y. Takagaki, S. Hanaoka, M. Katsuki, M. Yokoyama, T. Hasegawa, S. Yamazaki, and N. Shinohara. 1994. Origin of a T cell clone with a mismatched combination of MHC restriction and coreceptor expression. J Immunol 153:4496-4507.
46. Shinohara, N., N. Hozumi, M. Watanabe, J. A. Bluestone, R. Johnson-Leva, and D. H. Sachs. 1988. Class II antigen-specific murine cytolytic T lymphocytes (CTL). II. Genuine class II specificity of Lyt-2+ CTL clones. J Immunol 140:30-36.
47. Watanabe, H., M. Fujiwara, T. Mashiko, and M. Ito. 1989. T-cell clones with L3T4-positive or Lyt-2-positive phenotypes responding to mutant MHC class II antigen and inducing graft versus host reaction. J Invest Dermatol 93:691-694.
48. Hioe, C. E., and V. S. Hinshaw. 1989. Induction and activity of class II-restricted, Lyt-2+ cytolytic T lymphocytes specific for the influenza H5 hemagglutinin. J Immunol 142:2482-2488.
49. Morrison, L. A., V. L. Braciale, and T. J. Braciale. 1985. Expression of H-2I region-restricted cytolytic activity by an Lyt-2+influenza virus-specific T lymphocyte clone. J Immunol 135:3691-3696.
50. Matechak, E. O., N. Killeen, S. M. Hedrick, and B. J. Fowlkes. 1996. MHC class II-specific T cells can develop in the CD8 lineage when CD4 is absent. Immunity 4:337-347.
51. Martin, P., A. Duran, S. Minguet, M. L. Gaspar, M. T. Diaz-Meco, P. Rennert, M. Leitges, and J. Moscat. 2002. Role of zeta PKC in B-cell signaling and function. EMBO J 21:4049-4057.
52. Hashimoto, A., K. Takeda, M. Inaba, M. Sekimata, T. Kaisho, S. Ikehara, Y. Homma, S. Akira, and T. Kurosaki. 2000. Cutting edge: essential role of phospholipase C-gamma 2 in B cell development and function. J Immunol 165:1738-1742.
53. Smith-Garvin, J. E., G. A. Koretzky, and M. S. Jordan. 2009. T cell activation. Annu Rev Immunol 27:591-619.
54. Kroll, J., X. Shi, A. Caprioli, H. H. Liu, C. Waskow, K. M. Lin, T. Miyazaki, H. R. Rodewald, and T. N. Sato. 2005. The BTB-kelch protein KLHL6 is involved in B-lymphocyte antigen receptor signaling and germinal center formation. Mol Cell Biol 25:8531-8540.
55. Zheng, Y., H. Liu, J. Coughlin, J. Zheng, L. Li, and J. C. Stone. 2005. Phosphorylation of

RasGRP3 on threonine 133 provides a mechanistic link between PKC and Ras signaling systems in B cells. Blood 105:3648-3654.
56. Veldhoen, M., K. Hirota, J. Christensen, A. O'Garra, and B. Stockinger. 2009. Natural agonists for aryl hydrocarbon receptor in culture medium are essential for optimal differentiation of Th17 T cells. J Exp Med 206:43-49.
57. Svensson, C., A. E. Silverstone, Z. W. Lai, and K. Lundberg. 2002. Dioxin-induced adseverin expression in the mouse thymus is strictly regulated and dependent on the aryl hydrocarbon receptor. Biochem Biophys Res Commun 291:1194-1200.
58. Vogel, C. F., E. Sciullo, W. Li, P. Wong, G. Lazennec, and F. Matsumura. 2007. RelB, a new partner of aryl hydrocarbon receptor-mediated transcription. Mol Endocrinol 21:2941-2955.
59. Yen, H. R., T. J. Harris, S. Wada, J. F. Grosso, D. Getnet, M. V. Goldberg, K. L. Liang, T. C. Bruno, K. J. Pyle, S. L. Chan, R. A. Anders, C. L. Trimble, A. J. Adler, T. Y. Lin, D. M. Pardoll, C. T. Huang, and C. G. Drake. 2009. Tc17 CD8 T cells: functional plasticity and subset diversity. J Immunol 183:7161-7168.
60. Sharma, L. C., N. Muirhead, and A. I. Lazarovits. 1997. Human mouse chimeric CD7 monoclonal antibody (SDZCHH380) for the prophylaxis of kidney transplant rejection: analysis beyond 4 years. Transplant Proc 29:323-324.
61. Sun, Z., C. W. Arendt, W. Ellmeier, E. M. Schaeffer, M. J. Sunshine, L. Gandhi, J. Annes, D. Petrzilka, A. Kupfer, P. L. Schwartzberg, and D. R. Littman. 2000. PKC-theta is required for TCR-induced NF-kappaB activation in mature but not immature T lymphocytes. Nature 404:402-407.
62. Bauer, B., N. Krumbock, N. Ghaffari-Tabrizi, S. Kampfer, A. Villunger, M. Wilda, H. Hameister, G. Utermann, M. Leitges, F. Uberall, and G. Baier. 2000. T cell expressed PKCtheta demonstrates cell-type selective function. Eur J Immunol 30:3645-3654.
63. Rott, O., J. Charreire, M. Semichon, G. Bismuth, and E. Cash. 1995. B cell superstimulatory influenza virus (H2-subtype) induces B cell proliferation by a PKC-activating, Ca(2+)-independent mechanism. J Immunol 154:2092-2103.

1-38. (canceled)

39. A method, comprising the steps of:
recovering T-cells from a mammalian host previously transplanted with an allograft after transplantation of the allograft;
culturing the T-cells on a base of semi-professional antigen presenting cells; and
selecting at least one CD8 T-cell that expands on the base of semi-professional antigen presenting cells, wherein the at least one CD8 T-cell is resistant to an inhibitor selected from the group consisting of a calcineurin inhibitor and a mTOR inhibitor.

40. The method of claim 39, wherein the step of culturing is performed by culturing the T-cells on the base of semi-professional antigen presenting cells selected from the group consisting of smooth muscle cells, endothelial cells and epithelial cells.

41. The method of claim 39, wherein the step if selecting is performed by selecting at least one CD8 T-cell that is resistant to the inhibitor selected from the group consisting of cyclosporine, tacrolimus, and rapamycin.

42. The method of claim 39, further comprising the steps of:
placing the at least one CD8 T-cell in contact with a concentration of the inhibitor, the concentration sufficient to inhibit growth of traditional CD8 T-cell not cultured on a base of semi-professional antigen presenting cells; and
harvesting at least a second CD8 T-cell that proliferates after being in contact with the concentration of the inhibitor.

43. The method of claim 39, further comprising step of:
placing the at least one CD8 T-cell in contact with an antibody capable of selectively binding to IL18r1.

44. The method of claim 42, further comprising the step of:
testing the at least one second CD8 T-cell to determine if the at least one second CD8 T-cell expresses at least one gene selected from the group consisting of Mest, Padi2, Ahr, Klh16, Rasgrp3, Klhi30, Trib2, Rab17, Prkcz, PLCγ2, Scin, Pla2g4a, CD7, IL18r1, IL-17a, IL-17f, Sgk3, Gpr15, Pls3, and Zfp187.

45. The method of claim 39, wherein the at least one CD8 T-cell is CD8bm12-1 or an equivalent cell type.

46. A method, comprising the steps of:
obtaining a sample containing at least one CD8 T-cell from a mammal that has previously or is currently rejecting an allograft transplant; and
placing the sample in contact with a first antibody capable of selectively binding to a second antibody, the second antibody selected from the group consisting of at least one of an anti-CD7 antibody and an anti-IL18r1 antibody; and
collecting at least a second CD8 T-cell that expands after the at least one CD8 T-cell is placed in contact with the first antibody, wherein the at least a second CD8 T-cell is resistant to an inhibitor selected from the group consisting of a calcineurin inhibitor and a mTOR inhibitor.

47. The method of claim 46, wherein the at least a second CD8 T-cell is bound to the first antibody, and wherein the first antibody is bound to the selected second antibody.

48. The method of claim 46, wherein the at least a second CD8 T-cell is CD8bm12-1 or an equivalent cell type.

49. A method for selecting for a compound to regulate a subset of CD8 T-cells, the method comprising the steps of:
placing a population of CD8 T-cells in contact with at least one compound, wherein the CD8 T-cells express Ahr and are resistant to an inhibitor selected from the group consisting of a calcineurin inhibitor and a mTOR inhibitor;
measuring an effect of the compound on the population of CD8 T-cells; and
choosing a desired compound from the at least one compound, the desired compound capable of regulating a subset of CD8 T-cells.

50. The method of claim 49, wherein the placing step is performed to place the population of CD8 T-cells in contact with the at least one compound in vitro or in vivo.

51. The method of claim 49, wherein at least one of the at least one compound is/are selected from a group of compounds that inhibit the expression or function of at least one gene selected from the group consisting of Mest, Padi2, Ahr, Klh16, Rasgrp3, Klhi30, Trib2, Rab17, Prkcz, PLCγ2, Scin, Pla2g4a, CD7, IL18r1, IL-17a, IL-17f, Sgk3, Gpr15, P1s3, and Zfp187.

52. The method of claim 49, wherein the compound is selected from the group consisting of a siRNA and an antibody.

53. The method according to claim 49, wherein at least one of the at least one compound is/are selected from a group of compounds that preferentially binds to at least one gene product encoded by at least one gene selected from the group consisting of Mest, Padi2, Ahr, Klh16, Rasgrp3, Klhi30, Trib2, Rab17, Prkcz, PLCγ2, Scin, Pla2g4a, CD7, IL18r1, IL-17a, IL-17f, Sgk3, Gpr15, P1s3, and Zfp187.

54. A method, comprising the steps of:
administering at least one therapeutically effective dose of at least one compound to a patient that has undergone an allograft transplant, the at least one compound capable of inhibiting proliferation of a subset of CD8 T-cells that express Ahr and that are resistant to an inhibitor selected from the group consisting of a calcineurin inhibitor and a mTOR inhibitor.

55. The method of claim 54, wherein at least one of the at least one compound is selected from a group consisting of a polyclonal antibody that preferentially bids to at least one of a calcineurin-resistant CD8 T-cell and a mTOR-resistant CD8 T-cell and (ii) a monoclonal antibody that preferentially bids to at least one of a calcineurin-resistant CD8 T-cell and a mTOR-resistant CD8 T-cell.

56. The method of claim 54, wherein at least one of the at least one compound is selected from an anti-CD7 antibody and an anti-IL18r1 receptor antibody.

57. The method of claim 54, wherein the at least one compound interacts with a product of at least one gene selected from the group consisting of Mest, Padi2, Ahr, Klh16, Rasgrp3, Klhi30, Trib2, Rab17, Prkcz, PLCγ2, Scin, Pla2g4a, CD7, IL18r1, IL-17a, IL-17f, Sgk3, Gpr15, Pls3, and Zfp187.

58. The method of claim 54, wherein the step of administering is performed to treat the patient experiencing allograft vasculopathy or chronic allograft rejection.


SEQUENCE LISTING (1 sequence)

SEQ ID NO: 1 - protein, 10 aa, Artificial Sequence
  predicted I-A bm12-specific peptide
EYWNSQPEFL